(19) United States
(12) Patent Application Publication
(10) Pub. No.: US 2025/0208141 A1
(43) Pub. Date: Jun. 26, 2025

(54) FLUORESCENT PROBE FOR LABELING M1 MACROPHAGES, AND M1 MACROPHAGE MONITORING SYSTEM USING SAME

(71) Applicants: INSTITUTE FOR BASIC SCIENCE, Daejeon (KR); POSTECH Research and Business Development Foundation, Pohang-si, Gyeongsangbuk-do (KR)

(72) Inventors: Young-Tae CHANG, Busan (KR); Haw-Young KWON, Pohang-si (KR); Heewon CHO, Seoul (KR)

(73) Assignees: INSTITUTE FOR BASIC SCIENCE, Daejeon (KR); POSTECH Research and Business Development Foundation, Pohang-si, Gyeongsangbuk-do (KR)

(21) Appl. No.: 18/852,010

(22) PCT Filed: Mar. 30, 2023
(86) PCT No.: PCT/KR2023/004294

§ 371 Date: Sep. 27, 2024

(30) Foreign Application Priority Data
Mar. 30, 2022 (KR) 10-2022-0039430
Mar. 29, 2023 (KR) 10-2023-0041236

(51) Int. Cl. G01N 33/58; C07H 15/26; C07H 23/00; G01N 21/64

(52) U.S. Cl. CPC G01N 33/582; C07H 15/26; C07H 23/00; G01N 21/6428; G01N 2021/6439; G01N 2800/24

(57) ABSTRACT

The present disclosure relates to: a novel compound comprising carbohydrate derivatives and fluorophores; a fluorescent probe for labeling M1 macrophages comprising same; and a visualization composition and kit comprising the fluorescent probe. A fluorescent probe for labeling M1 macrophages, according to one embodiment, shows excellent selectivity for M1 macrophages, and thus, the fluorescent probe can be applied to a M1 macrophage monitoring system, a method for providing information on M1 macrophages, and a method for providing information necessary for diagnosis of inflammatory diseases.

### TECHNICAL FIELD

The present disclosure relates to a fluorescent probe for labeling an M1 macrophage, and an M1 macrophage monitoring system using the same.

### BACKGROUND ART

A macrophage is an important immune cell and controls an immune response by protecting our body through cytokine secretion and binding to other immune cells. A distinguished characteristic of the macrophage is plasticity, and inactive macrophages (MO) move from blood circulating monocytes to a tissue in order to start an immune response, and then are polarized into two representative forms, M1 and M2, depending on an environment. The macrophages are generally classified into the following representative two polarization states: M1 (pro-inflammatory) and M2 (anti-inflammatory). Considering the plasticity of the macrophage, it is very important to develop a fluorescent probe which may sense a specific subset of the macrophage.

An M1 macrophage is characterized by producing a pro-inflammatory cytokine and show sterilization activity. However, constant hyperactivation of a pro-inflammatory function of the M1 macrophage leads to chronic inflammation which causes autoimmune diseases such as rheumatoid arthritis (Funes, S. C., Rios, M., Escobar-Vera, J. & Kalergis, A. M. Implications of macrophage polarization in autoimmunity. Immunology 154, 186-195 (2018)).

### DISCLOSURE

### Technical Problem

An object of the present disclosure is to provide a novel compound comprising a carbohydrate derivative and a fluorophore.

Another object of the present disclosure is to provide a fluorescent probe for labeling an M1 macrophage comprising the compound.

Another object of the present disclosure is to provide a composition for visualization and a kit for visualization of an M1 macrophage comprising the fluorescent probe according to an example embodiment.

Another object of the present disclosure is to provide an M1 macrophage monitoring system comprising the fluorescent probe according to an example embodiment.

Another object of the present disclosure is to provide a method for providing information on an M1 macrophage by administering the composition comprising the fluorescent probe according to an example embodiment to a biological sample.

Still another object of the present disclosure is to provide a method for providing information needed to diagnose an inflammatory disease using the fluorescent probe according to an example embodiment.

### Technical Solution

In one general aspect, a compound represented by the following Chemical Formula 1 or a hydrate thereof is provided:

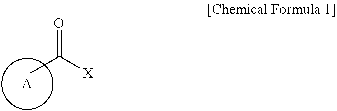

wherein
A is a carbohydrate derivative represented by the following Chemical Formula 2 or Chemical Formula 3:

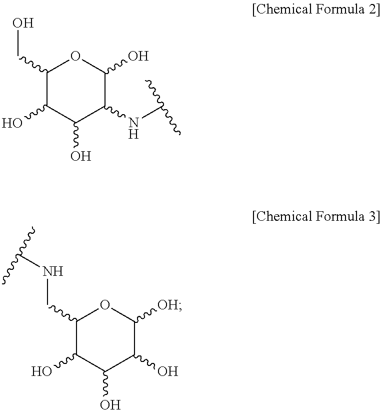 [SMILES: *NC1C(O)OC(CO)C(O)C1O ; *NCC1OC(O)C(O)C(O)C1O]

and
X is a fluorophore represented by any one of the following Chemical Formula 4 to Chemical Formula 7:

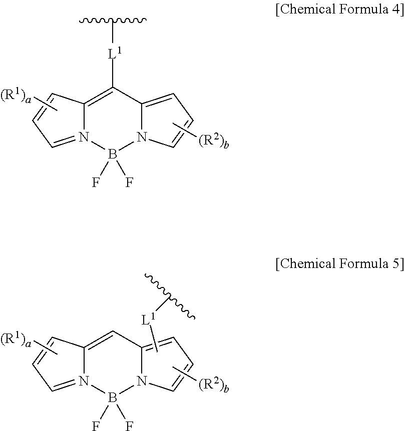

wherein
L¹ is independently of each other C₁₋₁₀ alkylene;
R¹ is independently of each other hydrogen, C₁₋₁₀ alkyl, or

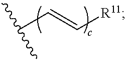

R² is independently of each other hydrogen, C₁₋₁₀ alkyl, or

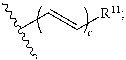

R¹¹ of R¹ and R² is independently of each other C₆₋₂₀ aryl, and the C₆₋₂₀ aryl may be substituted by one or more substituents selected from the group consisting of halogen, C₁₋₅ alkyl, and C₁₋₅ alkoxy;
c is independently of each other an integer of 0 to 10; and
a and b are independently of each other an integer of 1 to 3;

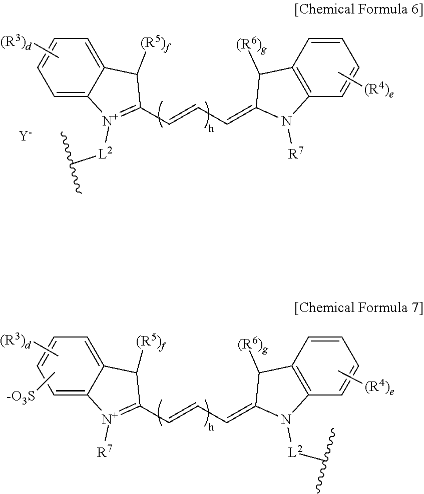

wherein
L² is independently of each other C₁₋₁₀ alkylene;
R³ and R⁴ are independently of each other hydrogen, halogen, C₁₋₁₀ alkyl, or C₁₋₁₀ alkoxy;
R⁵ and R⁶ are independently of each other hydrogen or C₁₋₁₀ alkyl;
R⁷ is independently of each other hydrogen or C₁₋₁₀ alkyl;
d is independently of each other an integer of 1 to 3;
e is independently of each other an integer of 1 to 4;
f and g are independently of each other 1 or 2;
h is independently of each other an integer of 0 to 10; and
Y⁻ is an anion.

In another general aspect, a fluorescent probe for labeling an M1 macrophage comprises the compound or the hydrate thereof according to an example embodiment.

In another general aspect, a composition for visualization of an M1 macrophage comprises the fluorescent probe for labeling an M1 macrophage according to an example embodiment.

In another general aspect, a kit for visualization of an M1 macrophage comprises the fluorescent probe for labeling an M1 macrophage according to an example embodiment.

In another general aspect, a method for providing information n M1 macrophage by administrating a composition comprising the fluorescent probe for labeling an M1 macrophage according to an example embodiment to a biological sample is provided.

In another general aspect, a method for providing information needed to diagnose an inflammatory disease comprises: obtaining a fluorescence image, by bringing a biological sample separated from a subject into contact with the fluorescent probe for labeling an M1 macrophage according to an example embodiment.

In still another general aspect, a method for diagnosing an inflammatory disease using the fluorescent probe for labeling an M1 macrophage according to an example embodiment is provided.

### Advantageous Effects

The present disclosure relates to a novel compound comprising a carbohydrate derivative and a fluorophore, a fluorescent probe for labeling an M1 macrophage comprising the same, and a composition and a kit for visualization comprising the fluorescent probe. Since the fluorescent probe for labeling an M1 macrophage according to an example embodiment shows excellent selectivity for an M1 macrophage, the fluorescent probe may be applied to an M1 macrophage monitoring system, a method for providing information on an M1 macrophage, and a method for providing information needed to diagnose an inflammatory disease.

### DESCRIPTION OF DRAWINGS

FIG. 1 is a drawing showing metabolic pathways of M1 and M2 macrophages. An M1 macrophage upregulates glucose transporters (GLUTs) in order to absorb a carbohydrate and operate aerobic glycolysis, while a M2 macrophage produces energy from OXOPHOS and β-oxidation by expressing CD36 highly.

FIG. 2 is a drawing showing results of a glucose (D/L-glucose) competitive test using CDr17.

FIG. 3 is a drawing showing results of performing a CDr17 absorption inhibition test through GLUT using cytochalasin B19 which is a GLUT inhibitor.

FIG. 4 is a drawing showing results of analyzing known 13 mouse (RAW264.7) GLUT gene expression levels in M0, M1, and M2 macrophages.

FIGS. 5 and 6 are drawings showing a mechanism for monitoring an inhibition effect by blocking a GLUT1 function using STF-3121 which is a selective inhibitor of GLUT1 in the M1 macrophage and then treating with CDr17 to analyze the fluorescence intensity, and the results thereof.

FIGS. 7 and 8 are drawings showing a mechanism for monitoring an inhibition effect by performing GLUT1 KO (knockout) in the M1 macrophage through CRISPR/Cas9, treating with CDr17, and then analyzing the fluorescence intensity of CDr17, and the results thereof.

FIGS. 9 and 10 are drawings showing results of analyzing fluorescence intensities after treating M0, M1, and M2 macrophages with CDr17 at a concentration of 0.5 μM, and it was confirmed to be sufficient to distinguish the M1 macrophage from M0 and M2 macrophages with CDr17 at only a low concentration.

FIG. 11 is a drawing showing results of analyzing fluorescence intensities after treating M0, M1, and M2 macrophages with 2-NBDG which is a generally used fluorescent probe at various concentrations, and it was confirmed that a fluorescence signal was able to be observed only when the concentration of 2-NBDG was increased to 100 μM, and also, M1 and M2 macrophages were not able to be clearly distinguished at any concentration.

FIG. 12 is a drawing showing a flow chart of a collagen antibody cocktail test which was performed in order to confirm whether CDr17 may be applied to diagnosis of rheumatoid arthritis.

FIG. 13 is a drawing showing results of observing joint swelling and redness, 3 days after antibody injection (IV) and then 10 days after administrating LPS by intraperitoneal (IP) injection to a mouse in a collagen antibody cocktail test.

FIG. 14 is a drawing showing results of analyzing a correlation between a CDr17 intensity and a joint swelling grade, and it was confirmed therefrom that there is a positive correlation between the swelling grade and the CDr17 intensity.

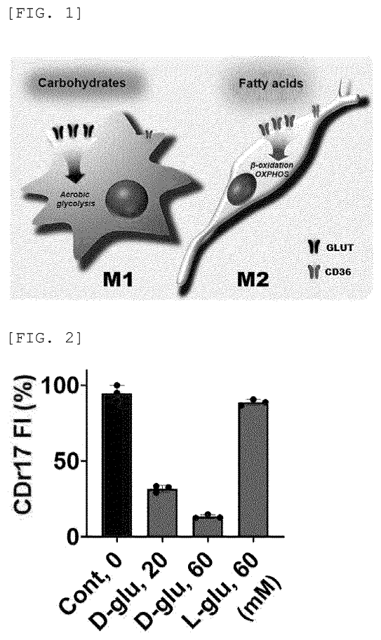

### BEST MODE

Since the embodiments described in the present specification may be modified in many different forms, the technology according to an example embodiment is not limited to the embodiments set forth herein. Furthermore, throughout the specification, unless otherwise particularly stated, the word “comprise”, “equipped”, “contain”, or “have” does not mean the exclusion of any other constituent element, but mean further inclusion of other constituent elements, and elements, materials, or processes which are not further listed are not excluded.

The numerical range used in the present specification comprises all values within the range comprising the lower limit and the upper limit, increments logically derived in a form and spanning in a defined range, all double limited values, and all possible combinations of the upper limit and the lower limit in the numerical range defined in different forms. As an example, when it is defined that a content of a composition is 10% to 80% or 20% to 50%, it should be interpreted that a numerical range of 10% to 50% or 50% to 80% is also described in the specification of the present specification. Unless otherwise defined in the present specification, values which may be outside a numerical range due to experimental error or rounding off of a value are also comprised in the defined numerical range.

Hereinafter, unless otherwise particularly defined in the present specification, “about” may be considered as a value within 30%, 25%, 20%, 15%, 10%, or 5% of a stated value.

The term “alkylene” used in the present specification refers to a diradical of a straight chain or branched chain of a carbon saturated bond, and may be substituted by any substituent.

The term “alkyl” used in the present specification refers to a radical of a straight chain or branched chain of a carbon saturated bond, and may be substituted by any substituent.

The term “aryl” used in the present specification may be a bridged ring, a spiro ring, or a fused ring without limitation. The aryl may be, for example, benzene, naphthalene, fluorene, anthracene, phenanthrene, bibenzene, triphenylene, pyrene, or chrisen.

The sign

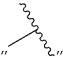

used in the present specification refers to a part in which a bond to other bonding groups is formed.

The sign “ [symbol] ” used in the present specification refers to “ [symbol] ” or “ [symbol] ” without limitation.

Each substituent defined “independently of each other” in the present specification may be the same or different. For example, in the case of each independent substituent (R¹) a, the a R¹'s may be the same as or different from each other.

Hereinafter, novel compound according to an example embodiment will be described in detail.

An example embodiment provides a compound represented by the following Chemical Formula 1 or a hydrate thereof:

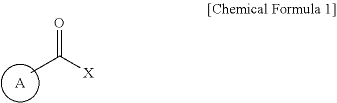

wherein
A is a carbohydrate derivative represented by the following Chemical Formula 2 or Chemical Formula 3:

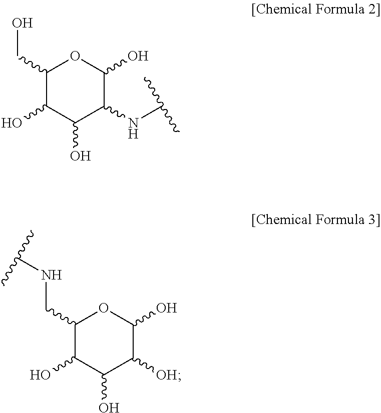 [SMILES: *NC1C(O)OC(CO)C(O)C1O ; *NCC1OC(O)C(O)C(O)C1O]

and
X is a fluorophore represented by any one of the following Chemical Formula 4 to Chemical Formula 7:

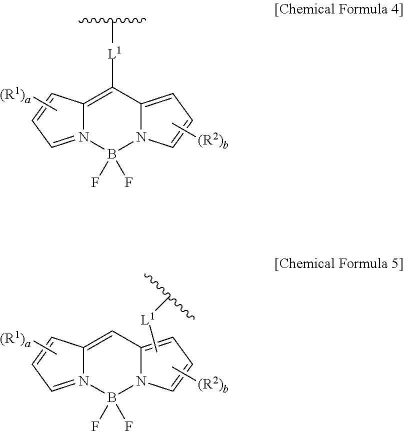

wherein
L¹ is independently of each other C₁₋₁₀ alkylene;
R¹ is independently of each other hydrogen, C₁₋₁₀ alkyl, or

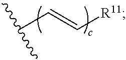

R² is independently of each other hydrogen, C₁₋₁₀ alkyl, or

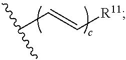

R¹¹ of R¹ and R² is independently of each other C₆₋₂₀ aryl, and the C₆₋₂₀ aryl may be substituted by one or more substituents selected from the group consisting of halogen, C₁₋₅ alkyl, and C₁₋₅ alkoxy;
c is independently of each other an integer of 0 to 10; and
a and b are independently of each other an integer of 1 to 3;

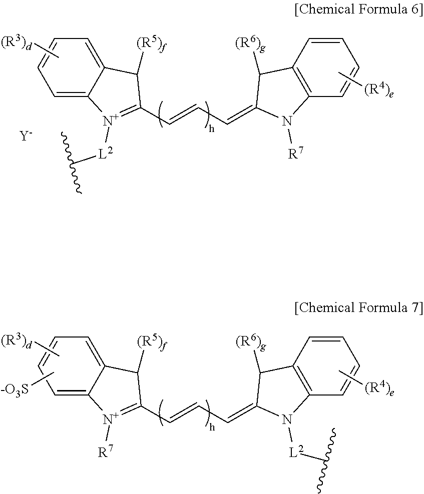

wherein
L² is independently of each other C₁₋₁₀ alkylene;
R³ and R⁴ are independently of each other hydrogen, halogen, C₁₋₁₀ alkyl, or C₁₋₁₀ alkoxy;
R⁵ and R⁶ are independently of each other hydrogen or C₁₋₁₀ alkyl;
R⁷ is independently of each other hydrogen or C₁₋₁₀ alkyl;
d is independently of each other an integer of 1 to 3;
e is independently of each other an integer of 1 to 4;
f and g are independently of each other 1 or 2;
h is independently of each other an integer of 0 to 10; and
Y⁻ is an anion.
   Since Y⁻ may be appropriately selected depending on the preparation method, it is not particularly limited, and may be selected from known organic or inorganic anions without limitation. For example, it may be a monovalent anion such as I⁻, Br⁻, Cl⁻, F⁻, NO₃⁻, ClO⁴⁻, PF⁶⁻, TFSI, BAFR, BF₄⁻, SO₃⁻, formate, acetate or a derivative thereof (for example, trifluoroacetate), triflate, sulfate, and phosphate. However, since the above is only an example of an anion, it is not necessarily limited to the above.

In an example embodiment, A may be a carbohydrate derivative represented by the following Chemical Formula 2-1, Chemical Formula 2-2, Chemical Formula 2-3, or Chemical Formula 3-1:

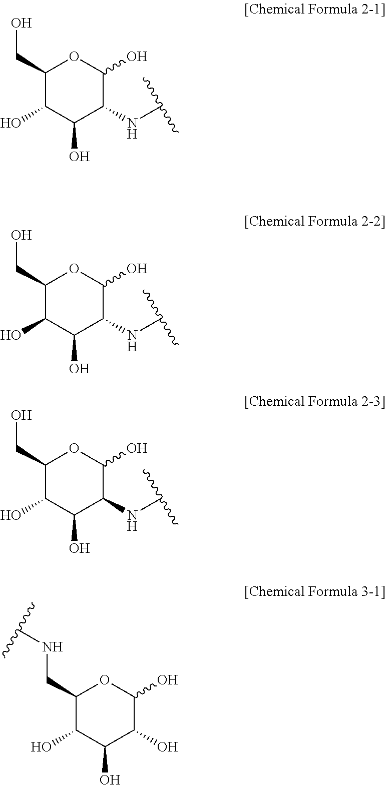

wherein
L¹ is independently of each other C₁₋₅ alkylene;
R¹ is independently of each other hydrogen, C₁₋₅ alkyl, or

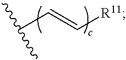

R² is independently of each other hydrogen, C₁₋₅ alkyl, or

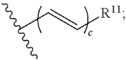

R¹¹ of R¹ and R² is independently of each other C₆₋₁₀ aryl, and the C₆₋₁₀ aryl may be substituted by one or more substituents selected from the group consisting of halogen, C₁₋₅ alkyl, and C₁₋₅ alkoxy;
c is independently of each other an integer of 1 to 5; and
a and b are independently of each other an integer of 1 to 3.

In an example embodiment, L¹ may be independently of each other C₁₋₃ alkylene or —CH₂CH₂—.

In an example embodiment, R¹ may be independently of each other hydrogen, C₁₋₃ alkyl, —CH₃, or

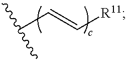

R¹¹ may be independently of each other C₆₋₁₀ aryl substituted by one or more substituents selected from the group consisting of halogen, C₁₋₃ alkyl, C₁₋₃ alkoxy, —CH₃, and —OCH₃, and c may be 1.

In an example embodiment, R¹ and R² may be independently of each other hydrogen, —CH₃,

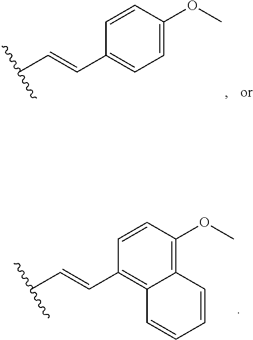 [SMILES: */C=C/c1ccc(OC)cc1 ; */C=C/c1ccc(OC)c2ccccc12]

In an example embodiment, Chemical Formula 4 may be a fluorophore represented by the following Chemical Formula 4-1, Chemical Formula 4-2, Chemical Formula 4-3, or Chemical Formula 4-4:

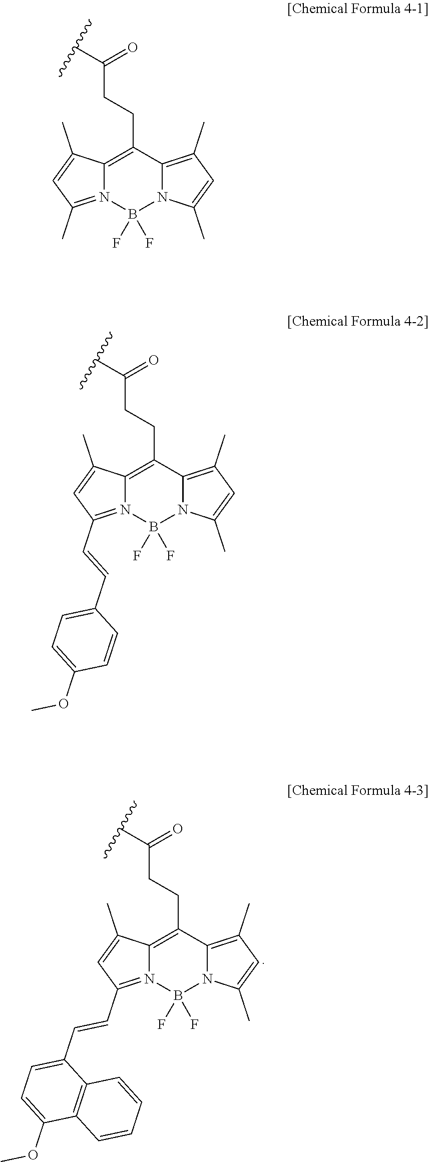

In an example embodiment, Chemical Formula 5 may be a fluorophore represented by the following Chemical Formula 5-1:

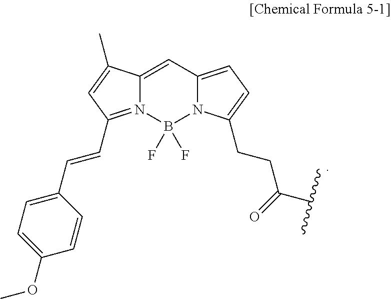

In an example embodiment, in Chemical Formula 6 and Chemical Formula 7,
L² may be independently of each other C₃₋₈ alkylene;
R³ and R⁴ may be independently of each other hydrogen or C₁₋₅ alkyl;
R⁵ and R⁶ may be independently of each other C₁₋₅ alkyl;
R⁷ may be independently of each other C₁₋₅ alkyl; and
i may be independently of each other an integer of 0 to 5.

In an example embodiment, R³ and R⁴ may be hydrogen.

In an example embodiment, R⁵ and R⁶ may be —CH₃.

In an example embodiment, Chemical Formula 6 may be a fluorophore represented by the following Chemical Formula 6-1 or Chemical Formula 6-2:

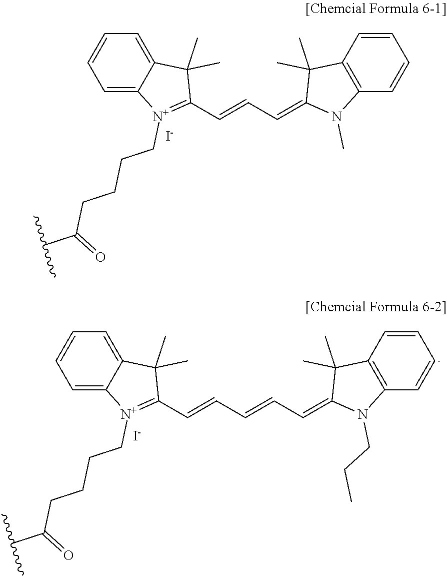 [SMILES: *C(=O)CCCC[N+]1=C(/C=C/C=C2/N(C)c3ccccc3C2(C)C)C(C)(C)c2ccccc21 ; [I-] ; *C(=O)CCCC[N+]1=C(/C=C/C=C/C=C2/N(CCC)c3ccccc3C2(C)C)C(C)(C)c2ccccc21 ; [I-]]

In an example embodiment, Chemical Formula 7 may be a fluorophore represented by the following Chemical Formula 7-1, Chemical Formula 7-2, or Chemical Formula 7-3:

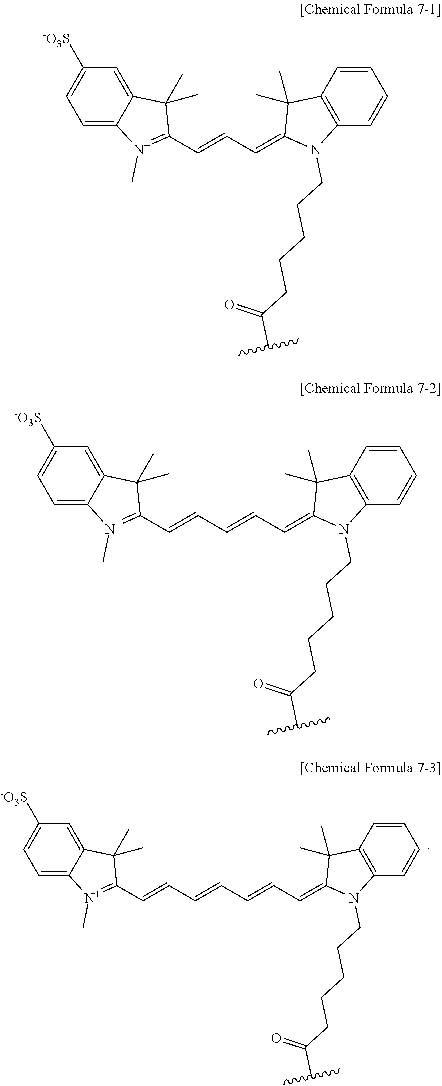

In an example embodiment, the compound represented by Chemical Formula 1 may be a compound selected from the following compound group, or a hydrate thereof:

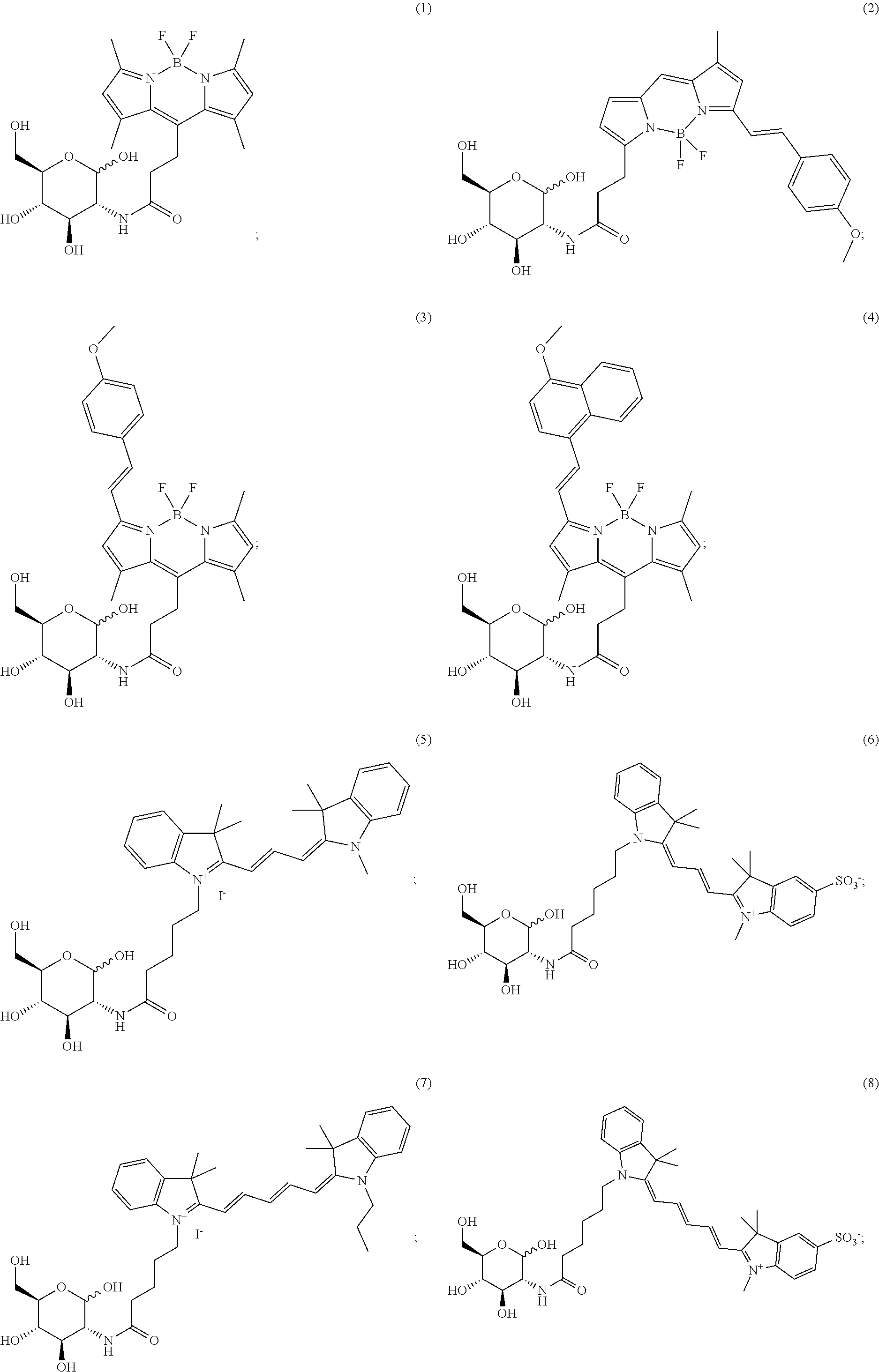

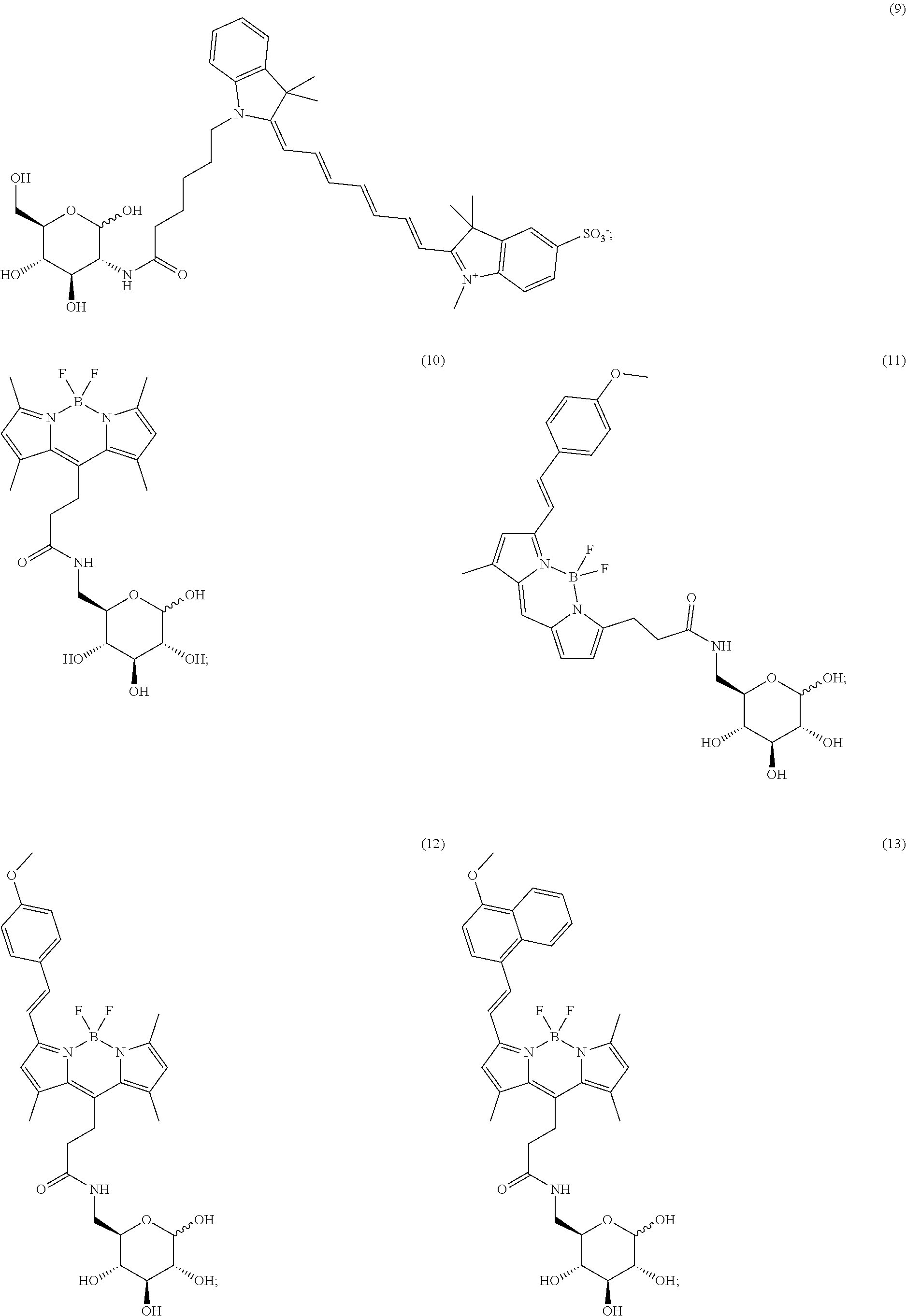

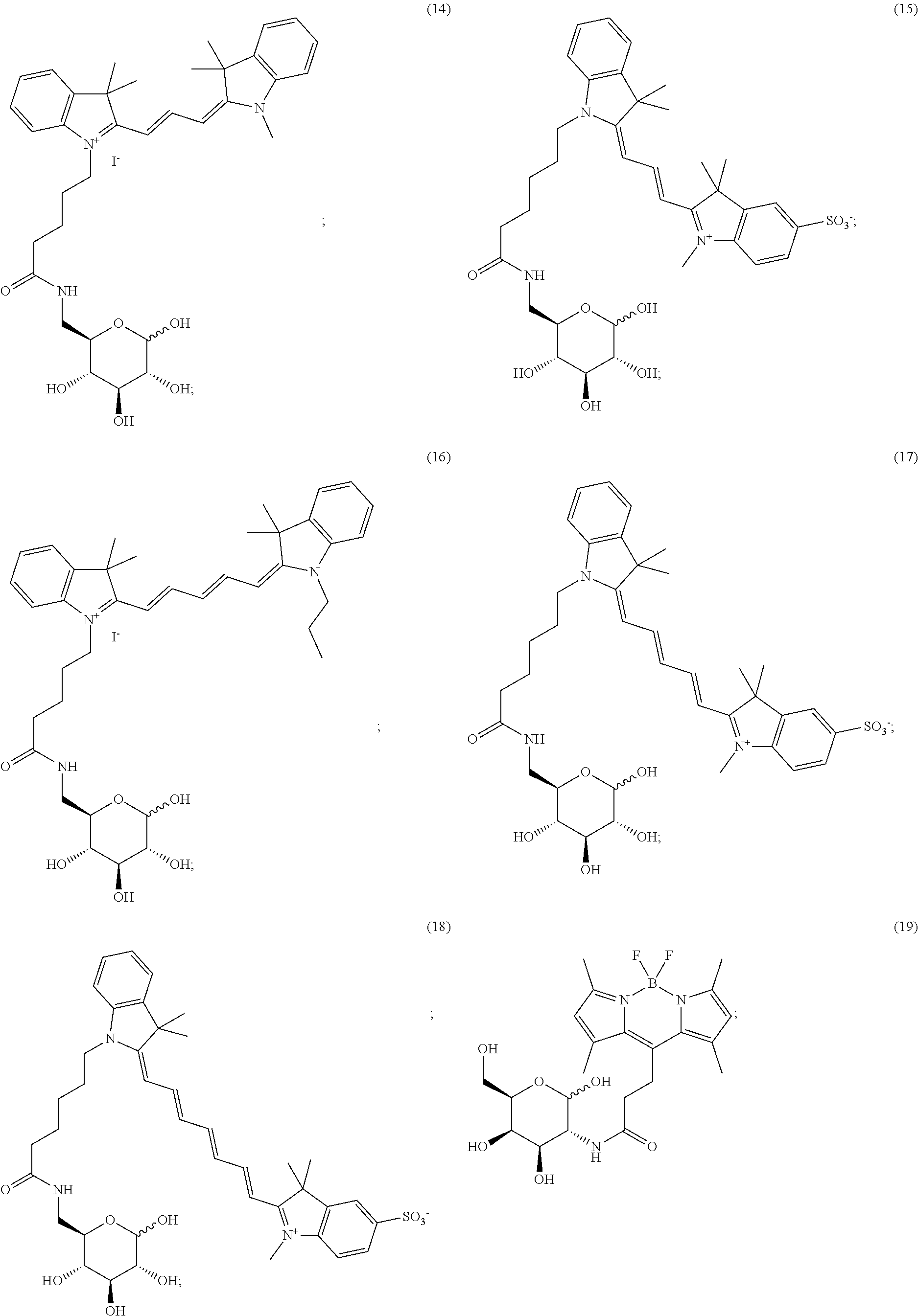

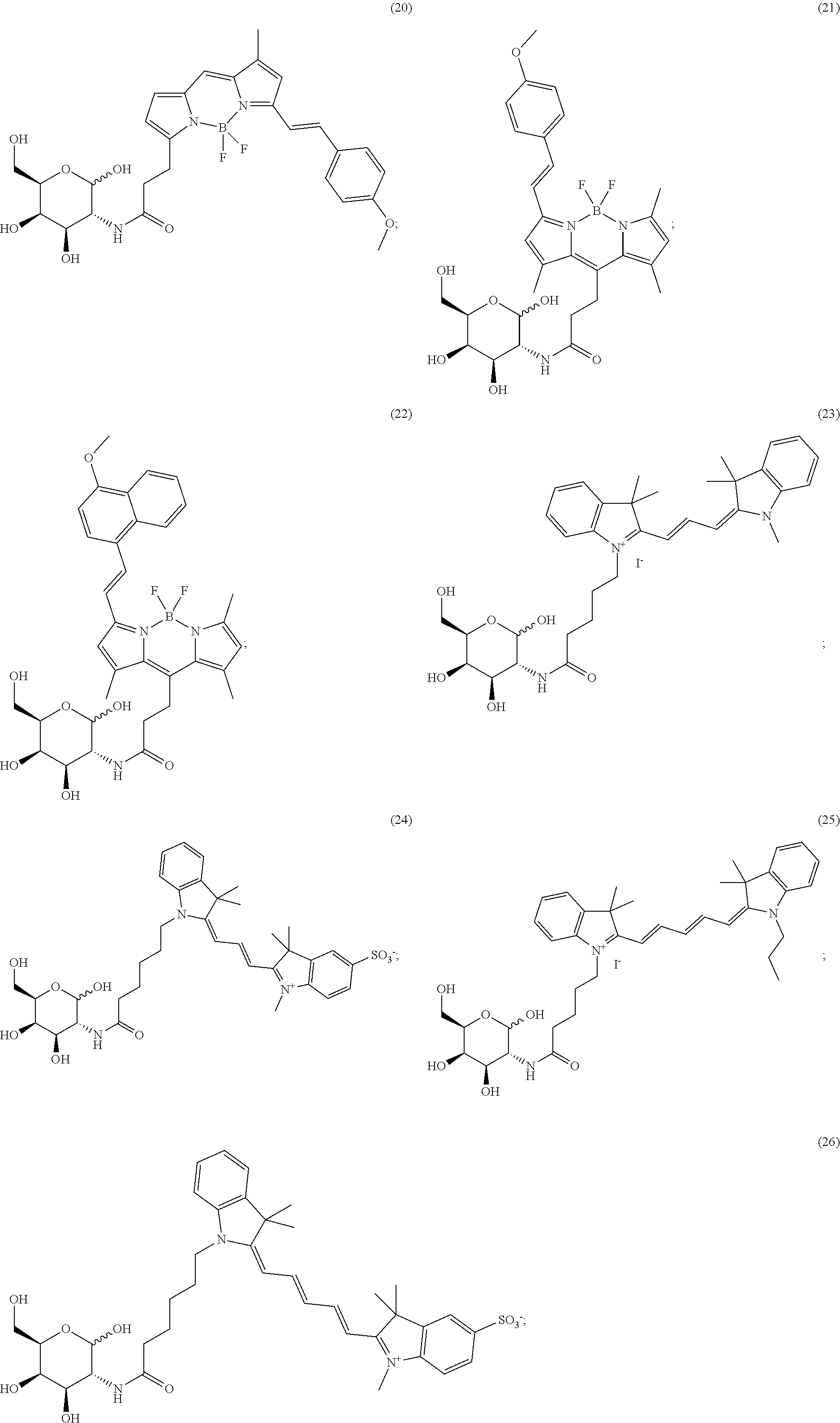

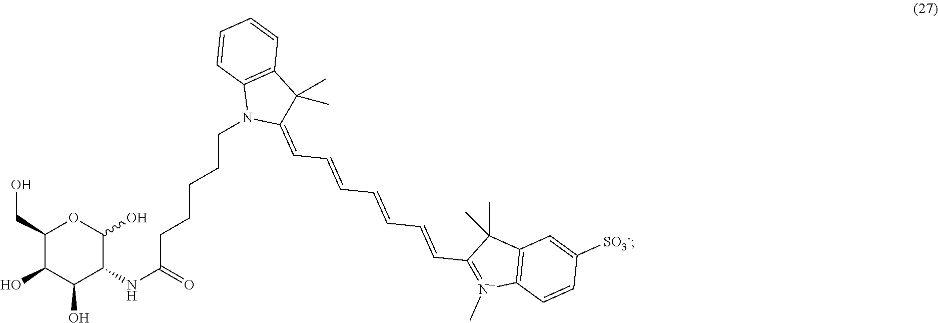

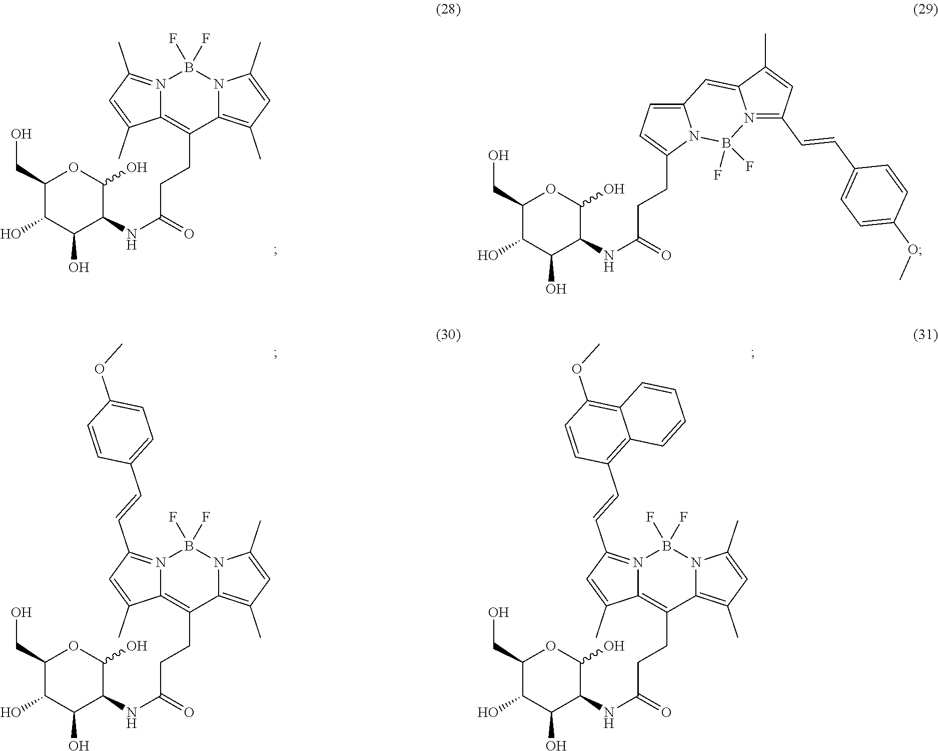

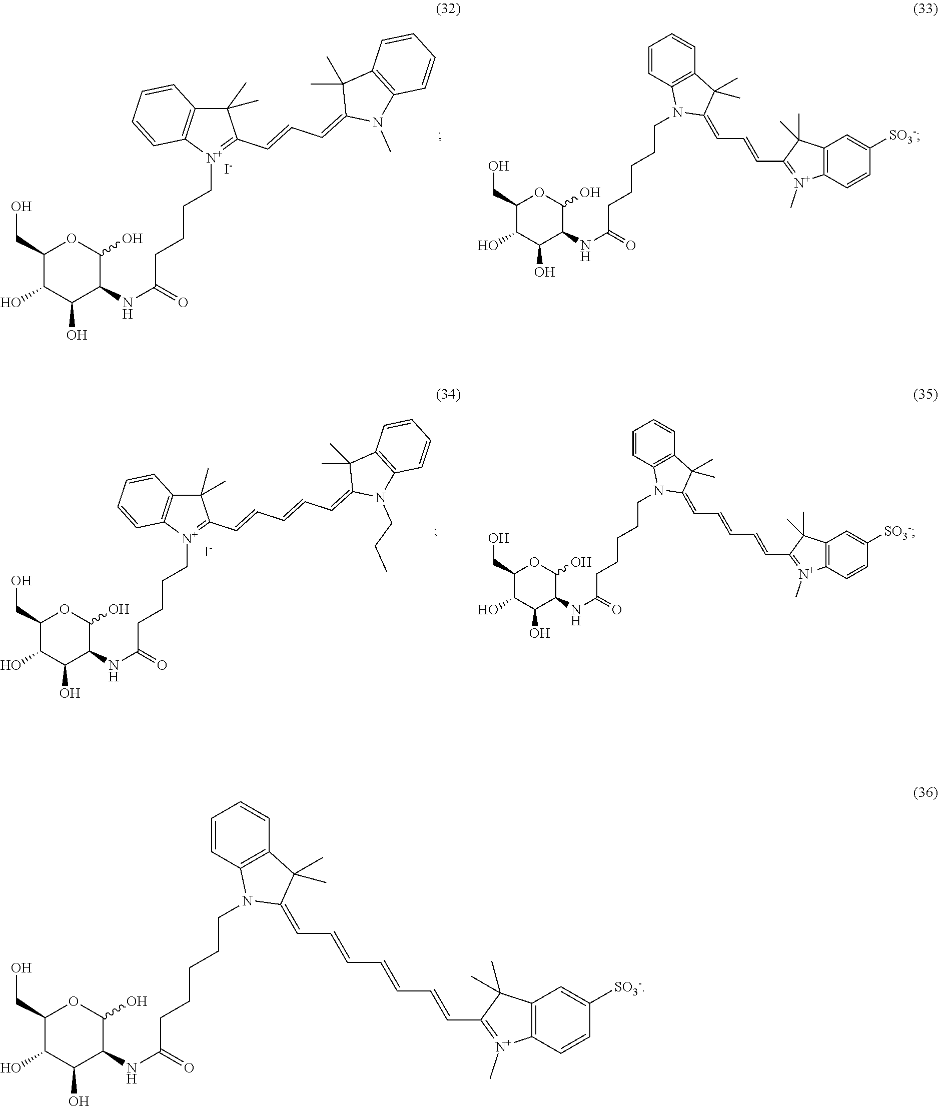

Another example embodiment provides a fluorescent probe for labeling an M1 macrophage comprising the compound represented by Chemical Formula 1 or the hydrate thereof according to an example embodiment.

The fluorescent probe for labeling an M1 macrophage according to an example embodiment targets glucose transporters (GLUTs). The fluorescent probe according to an example embodiment may target GLUT1 which is significantly expressed in the M1 macrophage.

The compound according to an example embodiment may show a difference in selectivity for the M1 macrophage depending on the structures of the carbohydrate derivative and the fluorophore. Specifically, a compound comprising a glucose derivative may show better selectivity than compounds comprising a galactose derivative or a mannose derivative. In addition, a cy5 group may show better selectivity than a cy3 group among the fluorophores according to the following Table 2.

The probe according to an example embodiment may be treated with a known dying reagent.

An example embodiment provides a composition for visualization and/or a kit for visualization of an M1 macrophage comprising the fluorescent probe for labeling an M1 macrophage according to an example embodiment. The composition for visualization and the kit for visualization are not necessarily limited to a specific use as long as they are a composition and a kit for visualizing an M1 macrophage using the fluorescent probe for labeling an M1 macrophage according to an example embodiment, and may be, for example, a composition for imaging or a kit for imaging. In addition, since an element which may be comprised in the composition for visualization and/or the kit for visualization of an M1 macrophage has no limitation in special configuration except for the fluorescent probe for labeling an M1 macrophage, a known constituent element may be further comprised.

Another example embodiment provides an M1 macrophage monitoring system comprising the fluorescent probe for labeling an M1 macrophage according to an example embodiment and a fatty acid transport protein.

Another example embodiment provides a method for providing information on an M1 macrophage by administrating a composition comprising the fluorescent probe for labeling an M1 macrophage according to an example embodiment to a biological sample. Specifically, an example embodiment provides a method for providing information on a position, an amount, and the like of an M1 macrophage.

Another example embodiment provides a method for providing information needed to diagnose an inflammatory disease comprising: obtaining a fluorescence image, by bringing a biological sample separated from a subject into contact with the fluorescent probe for labeling an M1 macrophage according to an example embodiment.

The information on the position, the amount, and the like of an M1 macrophage may be obtained by the step of obtaining of a fluorescent image, and the step may be a step of obtaining information by the position and/or the intensity of fluorescence.

In an example embodiment, the inflammatory disease may be, for example, an autoimmune disease, and the autoimmune disease may be, for example, multiple sclerosis, systemic lupus erythematosus, psoriasis, peripheral arthritis (in particular, psoriatic arthritis, rheumatoid arthritis, reactive arthritis, systemic juvenile idiopathic arthritis), axial arthritis (in particular, Bechterews disease), chronic inflammatory bowel disease (in particular, Crohn's disease, ulcerative colitis), atopic dermatitis, or allergic eczema/contact dermatitis.

### BEST MODE FOR CARRYING OUT THE INVENTION

Hereinafter, the examples and the experimental examples will be illustrated specifically in detail in the following. However, since the examples and the experimental examples described later only illustrate a part of one example embodiment, the technology described in the present specification should not be construed as being limited thereto.

### Examples 1 to 36 and Reference Examples 1 to 44

The compounds according to the examples and the reference examples are formed of the carbohydrate derivatives of the following Table 1 and the fluorophores of the following Table 2, and combinations of the specific carbohydrates and fluorophores of the compounds of the examples and the reference examples are shown in Table 3.

TABLE 1

| | | | | | | | |
|---|---|---|---|---|---|---|---|
| A | B | C | D | E | F | G | H |

| | | | |
|---|---|---|---|
| 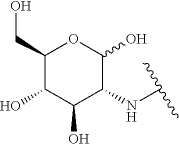 [SMILES: *N[C@H]1C(O)O[C@H](CO)[C@@H](O)[C@@H]1O] | 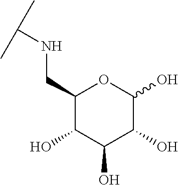 | 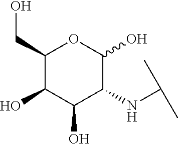 | 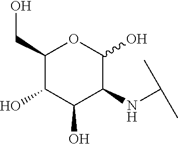 |

TABLE 2
c1

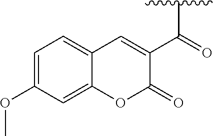 [SMILES: *C(=O)c1cc2ccc(OC)cc2oc1=O]

c2

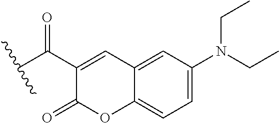 [SMILES: *C(=O)c1cc2cc(N(CC)CC)ccc2oc1=O]

c3

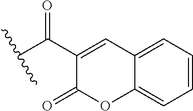 [SMILES: *C(=O)c1cc2ccccc2oc1=O]

c4

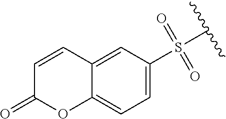 [SMILES: *S(=O)(=O)c1ccc2oc(=O)ccc2c1]

c5

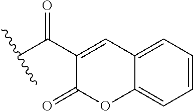 [SMILES: *C(=O)c1cc2ccccc2oc1=O]

c6

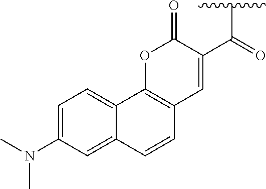 [SMILES: *C(=O)c1cc2ccc3cc(N(C)C)ccc3c2oc1=O]

n1

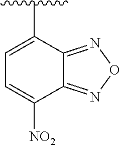 [SMILES: *c1ccc([N+](=O)[O-])c2nonc12]

n2

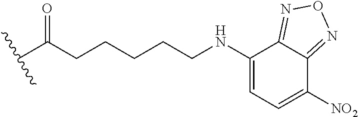 [SMILES: *C(=O)CCCCCNc1ccc([N+](=O)[O-])c2nonc12]

d1

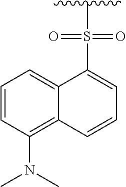 [SMILES: *S(=O)(=O)c1cccc2c(N(C)C)cccc12]

d2

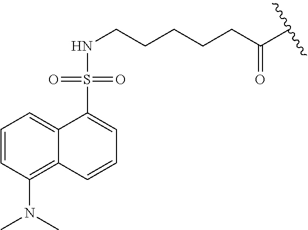 [SMILES: *C(=O)CCCCCNS(=O)(=O)c1cccc2c(N(C)C)cccc12]

f1

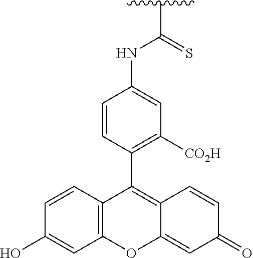 [SMILES: *C(=S)Nc1ccc(-c2c3ccc(=O)cc-3oc3cc(O)ccc23)c(C(=O)O)c1]

f2

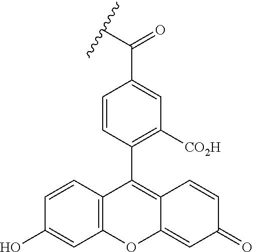 [SMILES: *C(=O)c1ccc(-c2c3ccc(=O)cc-3oc3cc(O)ccc23)c(C(=O)O)c1]

b1

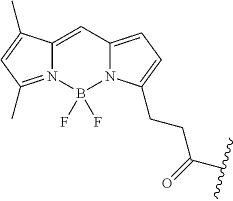

b2

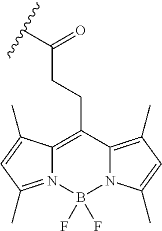

b3

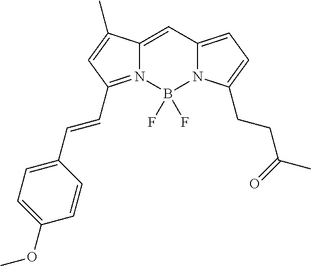

b4

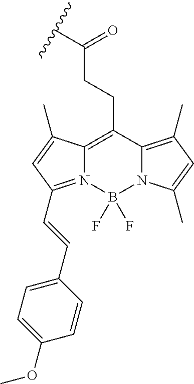

b5

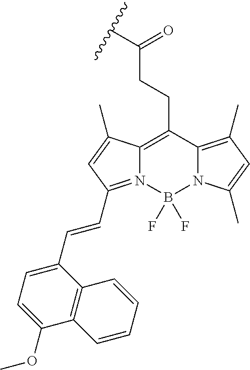

b6

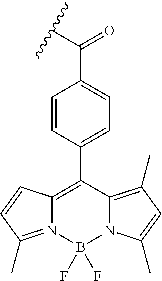

cy31

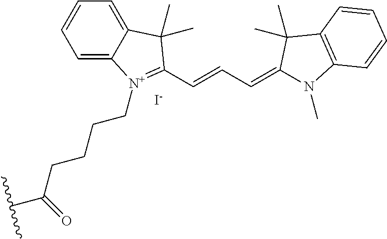 [SMILES: *C(=O)CCCC[N+]1=C(/C=C/C=C2/N(C)c3ccccc3C2(C)C)C(C)(C)c2ccccc21 ; [I-]]

cy32

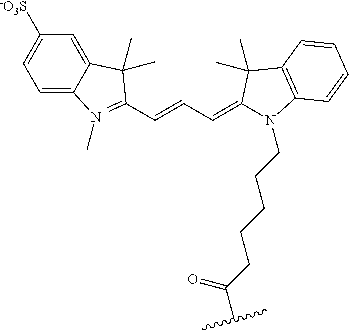

cy51

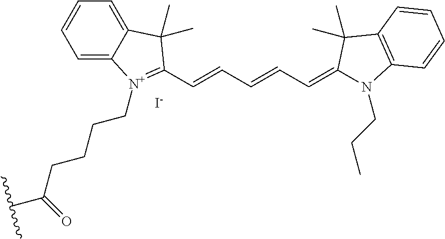 [SMILES: *C(=O)CCCC[N+]1=C(/C=C/C=C/C=C2/N(CCC)c3ccccc3C2(C)C)C(C)(C)c2ccccc21 ; [I-]]

cy52

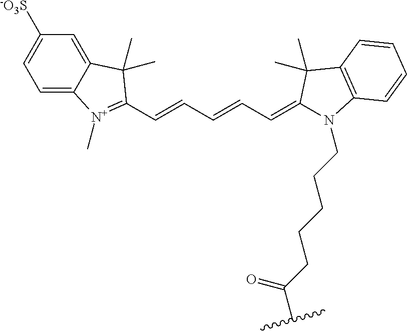

cy71

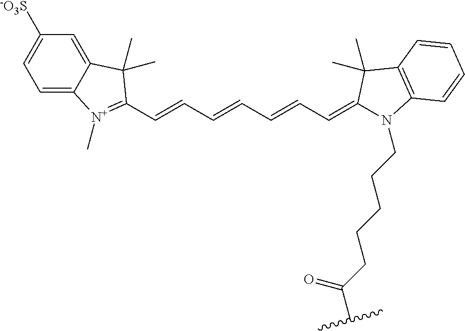

TABLE 3

| | 2 | 3 | 4 | 5 | 6 | 7 | 8 | 9 | 10 | 11 |
|---|---|---|---|---|---|---|---|---|---|---|
| A | c1 | c2 | c3 | n1 | n2 | d1 | d2 | f1 | f2 | b1 |
| | (Reference | (Reference | (Reference | (Reference | (Reference | (Reference | (Reference | (Reference | (Reference | (Reference |
| | Example 1) | Example 2) | Example 3) | Example 4) | Example 5) | Example 6) | Example 7) | Example 8) | Example 9) | Example 10) |
| B | b2 | b3 | b4 | b5 | b6 | cy31 | cy32 | cy51 | cy52 | cy71 |
| | (Exam- | (Exam- | (Exam- | (Exam- | (Reference | (Exam- | (Exam- | (Exam- | (Exam- | (Exam- |
| | ple 1) | ple 2) | ple 3) | ple 4) | Example 11) | ple 5) | ple 6) | ple 7) | ple 8) | ple 9) |
| C | c1 | c2 | c4 | c5 | c6 | n1 | n2 | d1 | d2 | f1 |
| | (Reference | (Reference | (Reference | (Reference | (Reference | (Reference | (Reference | (Reference | (Reference | (Reference |
| | Example 12) | Example 13) | Example 14) | Example 15) | Example 16) | Example 17) | Example 18) | Example 19) | Example 20) | Example 21) |
| D | b1 | b2 | b3 | b4 | b5 | cy31 | cy32 | cy51 | cy52 | cy71 |
| | (Reference | (Exam- | (Exam- | (Exam- | (Exam- | (Exam- | (Exam- | (Exam- | (Exam- | (Exam- |
| | Example 22) | ple 10) | ple 11) | ple 12) | ple 13) | ple 14) | ple 15) | ple 16) | ple 17) | ple 18) |
| E | c1 | c2 | c4 | c5 | c6 | n1 | n2 | d1 | d2 | f1 |
| | (Reference | (Reference | (Reference | (Reference | (Reference | (Reference | (Reference | (Reference | (Reference | (Reference |
| | Example 23) | Example 24) | Example 25) | Example 26) | Example 27) | Example 28) | Example 29) | Example 30) | Example 31) | Example 32) |
| F | b1 | b2 | b3 | b4 | b5 | cy31 | cy32 | cy51 | cy52 | cy71 |
| | (Reference | (Exam- | (Exam- | (Exam- | (Exam- | (Exam- | (Exam- | (Exam- | (Exam- | (Exam- |
| | Example 33) | ple 19) | ple 20) | ple 21) | ple 22) | ple 23) | ple 24) | ple 25) | ple 26) | ple 27) |
| G | c1 | c2 | c4 | c5 | c6 | n1 | n2 | d1 | d2 | f1 |
| | (Reference | (Reference | (Reference | (Reference | (Reference | (Reference | (Reference | (Reference | (Reference | (Reference |
| | Example 34) | Example 35) | Example 36) | Example 37) | Example 38) | Example 39) | Example 40) | Example 41) | Example 42) | Example 43) |
| H | b1 | b2 | b3 | b4 | b5 | cy31 | cy32 | cy51 | cy52 | cy71 |
| | (Reference | (Exam- | (Exam- | (Exam- | (Exam- | (Exam- | (Exam- | (Exam- | (Exam- | (Exam- |
| | Example 44) | ple 28) | ple 29) | ple 30) | ple 31) | ple 32) | ple 33) | ple 34) | ple 35) | ple 36) |

Among the compounds of the examples and the reference examples, representatively, the preparation method of Example 8 is as follows.

Glucosamine (10.7 mg, 0.03 mmole), cy51 (5 mg, 0.01 mmole), HATU (22.8 mg, 0.03 mmole), and DIEA (17.4 μL, 0.05 mmole) were dissolved in DMF (5 mL), stirring was performed for 2 hours, and the mixture was concentrated under vacuum. The residue was purified with silica gel chromatography using MeOH:DCM=1:15 to 1:5, and then further purified with HPLC to obtain a target compound as a blue solid (4.2 mg, 53%).

The remaining compounds the examples and the reference examples were able to be prepared referring to the preparation method of the compound of Example 8, and the compounds were prepared by selecting each starting compound instead of glucosamine and cy51 and appropriately adjusting the addition amount of a solvent and the like.

The LC-MS data of the compounds of the examples are summarized in Table 4.

TABLE 4

| | calcd. | | | | calcd. | | |
|---|---|---|---|---|---|---|---|
| | m/z | obs. m/z | Purity | | m/z | obs. m/z | Purity |
| No. | [M + H]⁺ | [M + H]⁺ | (%) | No. | [M + H]⁺ | [M + H]⁺ | (%) |
| 1 | 504.2 | 504.2 | 99 | 2 | 552.2 | 552.3 | 90 |
| 3 | 622.3 | 622.3 | 99 | 4 | 672.3 | 672.6 | 99 |
| 5 | 604.3 | 604.4 | 99 | 6 | 698.3 | 698.2 | 99 |
| 7 | 658.4 | 658.4 | 99 | 8 | 724.3 | 724.3 | 99 |
| 9 | 750.3 | 750.3 | 95 | 10 | 504.2 | 504.2 | 99 |
| 11 | 594.2 | 594.2 | 97 | 12 | 622.3 | 622.3 | 99 |
| 13 | 630.3 | 630.4 | 99 | 14 | 604.3 | 604.3 | 99 |
| 15 | 698.3 | 698.23 | 99 | 16 | 658.4 | 658.4 | 99 |
| 17 | 724.3 | 724.3 | 95 | 18 | 750.3 | 750.3 | 99 |
| 19 | 504.2 | 504.2 | 96 | 20 | 594.2 | 594.2 | 99 |
| 21 | 622.3 | 622.3 | 99 | 22 | 672.2 | 672.2 | 95 |
| 23 | 604.3 | 604.3 | 99 | 24 | 698.3 | 698.3 | 99 |
| 25 | 658.4 | 658.4 | 86 | 26 | 724.3 | 724.3 | 99 |
| 27 | 750.3 | 750.3 | 95 | 28 | 504.2 | 504.2 | 97 |
| 29 | 594.2 | 594.2 | 99 | 30 | 622.3 | 622.3 | 99 |
| 31 | 672.2 | 672.2 | 95 | 32 | 604.3 | 604.3 | 99 |
| 33 | 698.3 | 698.3 | 99 | 34 | 658.4 | 658.4 | 99 |
| 35 | 724.3 | 724.3 | 99 | 36 | 750.3 | 750.3 | 95 |

### <Experimental Example 1> Analysis of Fluorescence Intensity Heat Map

In order to analyze selectivity of the compounds prepared in the examples and the reference examples for an M1 macrophage, analysis was performed using a fluorescence intensity heat map, as follows. First, M1 and M2 macrophages were distinguished from a resting state M0 of RAW264.7 which is a mouse cell line. The M0, M1, and M2 macrophages were seeded in a 96-well plate, and treated with the above compounds. After culturing for 1 hour, a cell image was taken with an automated fluorescence microscope, the fluorescence intensity of each cell type was quantified, the selectivity index (SI) of M1 for M0 or M2 macrophage according to the following Equation 1 was calculated, and the results are shown as a heat map (Tables 5 and 6).

Table 5: ST1 of M1 for M0

Table 6: ST1 of M1 for M2

$$Selectivity index ( SI ) = \{ F ( M 1 ) - F ( M 0 or M 1 ) \} / \{ σ ( M 1 ) + σ ( M 0 or M 1 ) \} \qquad [ Equation 1 ]$$

wherein “F” is an average value of a fluorescence value obtained from a corresponding macrophage, and “σ” is a standard deviation value of a fluorescence value obtained from a corresponding macrophage.

TABLE 5

| | 2 | 3 | 4 | 5 | 6 | 7 | 8 | 9 | 10 | 11 |
|---|---|---|---|---|---|---|---|---|---|---|
| A | −1.7 | −1.6 | −1.4 | 0 | 0 | −1.7 | −0.9 | 0 | −0.5 | −1.6 |
| B | 1.3 | −0.8 | 1.1 | 1.6 | 0 | 1.1 | 2.0 | 3.4 | 2.6 | 2.5 |
| C | −1.2 | −1.1 | −1.5 | −1.4 | −1.2 | −0.4 | 0.1 | 0.4 | −0.4 | −0.2 |
| D | 0.5 | 0.9 | −0.6 | 1.5 | 1.5 | 1.2 | 2.9 | 2.7 | 1.2 | 2.3 |
| E | −1.1 | −0.8 | −1.7 | −2.1 | 0 | 0 | −2.0 | 0.7 | −1.3 | 0 |
| F | 0 | 1.1 | 0 | 2.1 | 0.9 | 1.7 | 1.5 | 2.6 | 1.3 | 2.6 |
| G | −2.3 | −2.1 | −1.6 | −1.9 | −1.7 | 0 | 0 | 0 | −1.6 | 1.4 |
| H | 0 | 0 | −0.3 | 0.9 | 1.0 | 2.6 | 2.6 | 2.3 | 2.4 | 2.5 |

TABLE 6

| | 2 | 3 | 4 | 5 | 6 | 7 | 8 | 9 | 10 | 11 |
|---|---|---|---|---|---|---|---|---|---|---|
| A | −0.6 | −1.2 | −0.6 | −0.7 | 1.0 | −1.9 | −3.0 | 0 | −0.2 | −1.9 |
| B | 1.3 | −0.9 | 1.1 | 2.7 | 0 | 1.6 | 1.8 | 3.0 | 2.7 | 2.5 |
| C | −0.4 | −0.5 | −0.7 | −0.2 | −0.8 | −0.6 | 1.7 | −0.4 | −0.8 | −0.1 |
| D | 0.3 | 1.2 | 0.1 | 1.4 | 2.4 | 1.0 | 2.5 | 2.7 | 0.9 | 1.7 |
| E | −0.3 | −1.2 | −1.2 | −0.9 | 0 | 0 | −1.5 | −0.6 | 0 | 0 |
| E | 0 | 0.9 | 0 | 2.3 | 1.7 | 1.4 | 1.1 | 2.6 | 1.4 | 2.1 |
| G | −1.4 | −1.3 | −0.5 | −0.7 | −1.1 | 0.7 | 0 | 0 | −3.5 | 0.2 |
| H | 0 | 0 | 1.2 | 1.0 | 1.5 | 1.6 | 2.1 | 2.3 | 2.5 | 2.5 |

As confirmed from Tables 5 and 6, the compounds according to the examples showed different activities depending on the type of carbohydrate and fluorophore, but showed significantly better selectivity for an M1 macrophage than the compound of the reference examples. Meanwhile, among the compounds of the examples, Example 8 formed of carbohydrate B and fluorophore Cy52 showed the most significant selectivity was named CDr17 (Compound Designation red 17).

### <Experimental Example 2> Analysis of Mechanism for M1 Macrophage Selectivity

In order to analyze a mechanism for selectivity of the compounds (probes) according to the examples, test was performed as follows, using the CDr17 compound of Example 8. First, in order to analyze whether the selectivity of CDr17 is related to GLUTs in cells, a glucose competition test was performed by the following method. As an M1 macrophage medium having no glucose differentiated in RAW264.7 cells, a medium comprising 10% Heat-Inactivated Fetal Bovine Serum (Gibco) and 1% penicillin streptomycin (WELGENE) comprising D/L-glucose at various concentrations (0 (Cont.), 20, 60 mM) were cultured for 5 minutes, and CDr17 (1 μM) was added. The nucleus was stained with Hoechst33342 (1 μg/mL), and the medium was replaced with a fresh medium after 30 minutes. Fluorescence microscopic imaging was performed with Operetta High Throughput screening with a 40× objective lens.

As a result of fluorescence intensity analysis, the intensity of CDr17 was decreased dose-dependently by D-glucose, but L-glucose did not affect absorption of CDr17 by the M1 macrophage (FIG. 2).

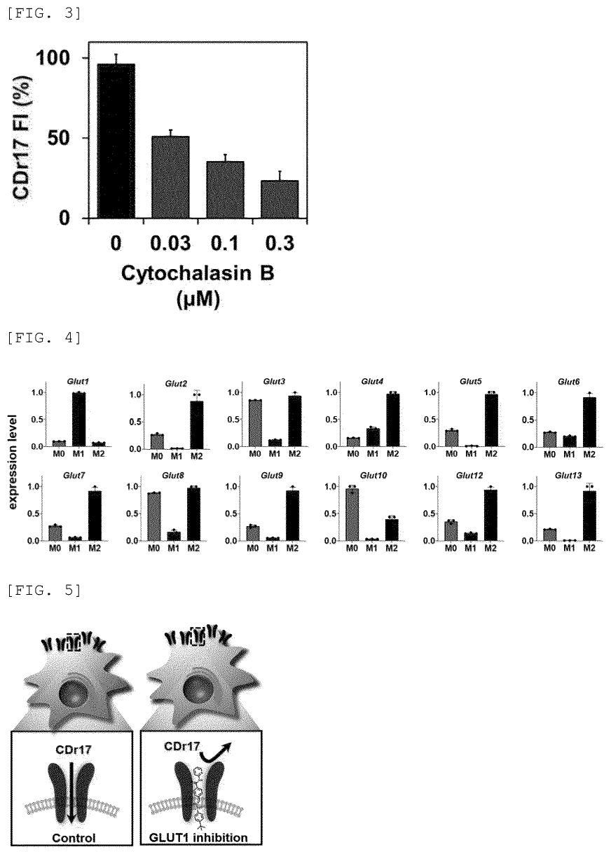

Next, a GLUT inhibition test was performed using cytochalasin B19 which is a general GLUT inhibitor. An inhibitor at various concentrations (0, 0.03, 0.1, 0.3 μM) was cultured for 30 minutes with the M1 macrophage, cells were treated with CDr17, and the fluorescence intensity was analyzed (FIG. 3). As a result, it was confirmed that the cytochalasin B19 blocked dose-dependently absorption of CDr17 through GLUT.

It was found that CDr17 acted as a substrate of GLUT like D-glucose from the test.

### <Experimental Example 3> Confirmation of Specific Target of CDr17

In order to specifically confirm the target range of CDr17, the test was performed as follows.

First, known 13 mouse (RAW264.7) GLUT gene expression levels in M0, M1, and M2 macrophages were analyzed, and as a result, it was confirmed that only GLUT1 was significantly expressed in M1 as compared with M0 and M2 macrophages (FIG. 4)

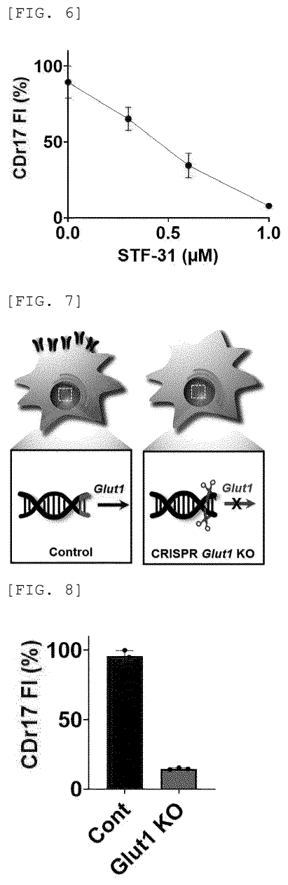

Next, the function of GLUT1 was blocked using STF-3121 which is a selective inhibitor of GLUT1 in the M1 macrophage of RAW264.7, and then a treatment was performed with 1 μM CDr17 for 30 minutes to monitor the inhibition effect. As a result, it was confirmed that entry of CDr17 into M1 cells was significantly decreased dependently on the dose of STF-31 (FIGS. 5 and 6).

Furthermore, GLUT1 knockout (KO) was performed in M1 macrophages through CRISPR/Cas9, and then a treatment with 1 μM CDr17 for 30 minutes to analyze the fluorescence intensity of CDr17. The knockout test was performed using M1 macrophages of RAW264.7 cells, and TrueCut™ Cas9 Protein v2 (Invitrogen™, A36498) and Lipofectamine™, CRISPRMAX™, Cas9 Transfection Reagent (Invitrogen™ CMAX00008) were used according to the guidelines of the manufacturer. sgRNA was purchased from Horizon and used (Target ID, SG-044254-01-0005). As a result of fluorescence intensity analysis, the intensity was decreased by 80% as compared with control M1 macrophages (FIGS. 7 and 8).

It was confirmed from the test that CDr17 targets GLUT1 in the M1 macrophages.

### <Experimental Example 4> Analysis of M1 Macrophage Selectivity by Comparison

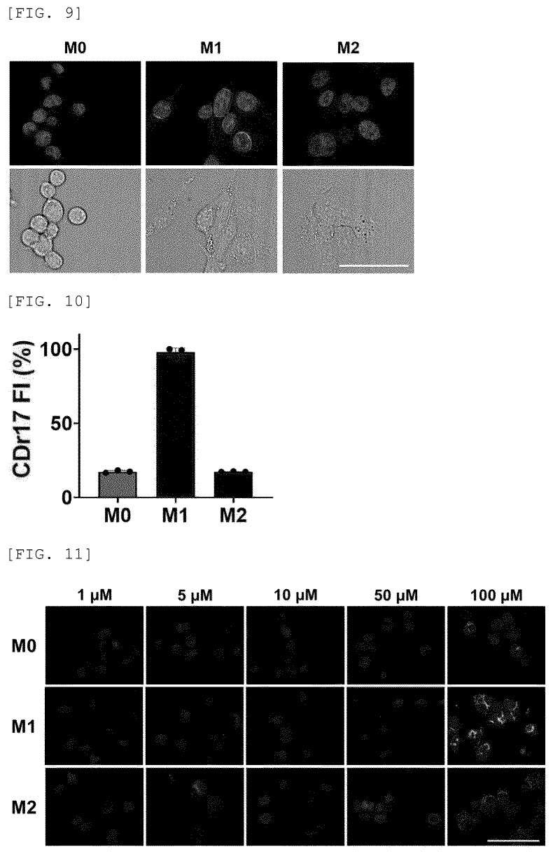

In order to compare the selectivity of CDr17 for M1 macrophages, 2-NBDG which is a generally used fluorescent probe was applied to THP-1 which is a macrophage cell line of human origin and tested. As a result of analyzing fluorescence intensity in each macrophage using CDr17 at a concentration of 0.5 μM and 2-NBDG at various concentrations (1, 5, 10, 50, 100 μM), it was sufficient to distinguish M0 and M2 macrophages from an M1 macrophage with CDr17 at a concentration of 0.5 μM (FIGS. 9 and 10), but with 2-NBDG, observation of fluorescence intensity was allowed only when the concentration was increased to 100 μM, and M1 and M2 macrophages were not clearly distinguished: any concentration (FIG. 11).

Thus, it was confirmed that CDr17 implemented excellent selectivity and universality for M1 macrophages.

### <Experimental Example 5> Diagnostic Analysis of Rheumatoid Arthritis Model

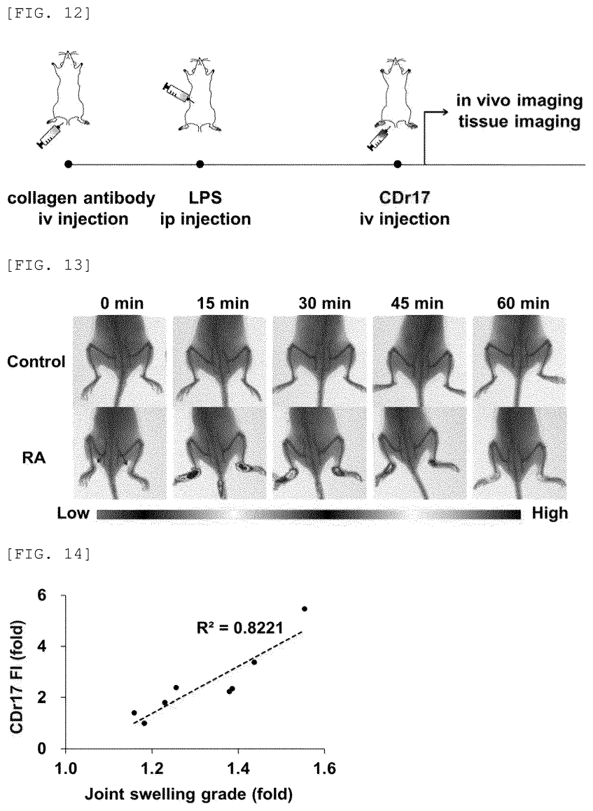

It is known that the macrophage of rheumatoid arthritis (RA) is formed of most M1s and a few M2s. In order to confirm whether CDr17 may be applied to analysis of rheumatoid arthritis, a test was performed using a collagen antibody cocktail (FIG. 12). Joint swelling and redness were observed 3 days after antibody injection (IV) and then 10 days after administrating LPS by intraperitoneal (IP) injection to a mouse (arrow in FIG. 13). CDr17 (2 mM, 100 μL) was injected through an IV, and then in-vivo images were collected at various points in time (0, 15, 30, 45, 60 minutes) (FIG. 13). As a result, the highest peak was shown when 15 minutes had passed, and it was confirmed that an inflammation area may be distinguished in a short time using CDr17.

Next, as a result of analyzing a correlation between a CDr17 intensity and a joint swelling grade, it was confirmed that there is a positive correlation between the swelling grade and the CDr17 intensity (FIG. 14), and it was confirmed therefrom that CDr17 may be used as a diagnosis probe which may tract the severity of the disease.

Hereinabove, though an example embodiment has been described in detail by the examples and the experimental examples, the scope of an example embodiment is not limited to specific examples, and should be construed by the appended claims.

1. A compound represented by the following Chemical Formula 1 or a hydrate thereof: 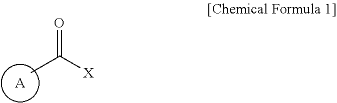
wherein
A is a carbohydrate derivative represented by the following Chemical Formula 2 or Chemical Formula 3: 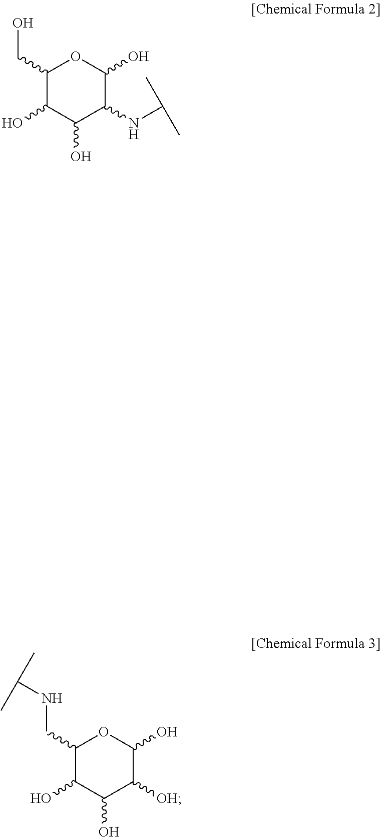
and
X is a fluorophore represented by any one of the following Chemical Formula 4 to Chemical Formula 7: 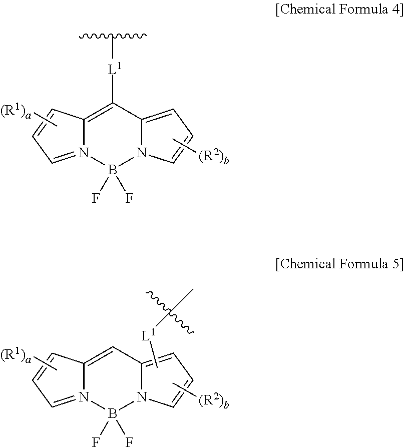
wherein
L¹ is independently of each other C₁₋₁₀ alkylene;
R¹ is independently of each other hydrogen, C₁₋₁₀ alkyl, or 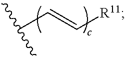
R² is independently of each other hydrogen, C₁₋₁₀ alkyl, or 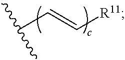
R¹¹ of R¹ and R² is independently of each other C₆₋₂₀ aryl, and the C₆₋₂₀ aryl may be substituted by one or more substituents selected from the group consisting of halogen, C₁₋₅ alkyl, and C₁₋₅ alkoxy;
c is independently of each other an integer of 0 to 10; and
a and b are independently of each other an integer of 1 to 3; 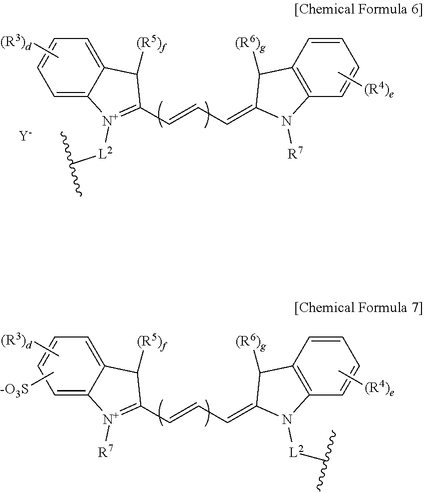
wherein
L² is independently of each other C₁₋₁₀ alkylene;
R³ and R⁴ are independently of each other hydrogen, halogen, C₁₋₁₀ alkyl, or C₁₋₁₀ alkoxy;
R⁵ and R⁶ are independently of each other hydrogen or C₁₋₁₀ alkyl;
R⁷ is independently of each other hydrogen or C₁₋₁₀ alkyl;
d is independently of each other an integer of 1 to 3;
e is independently of each other an integer of 1 to 4;
f and g are independently of each other 1 or 2;
h is independently of each other an integer of 0 to 10; and
Y⁻ is an anion.

2. The compound or the hydrate thereof of claim 1, wherein A is a carbohydrate derivative represented by the following Chemical Formula 2-1, Chemical Formula 2-2, Chemical Formula 2-3, or Chemical Formula 3-1:

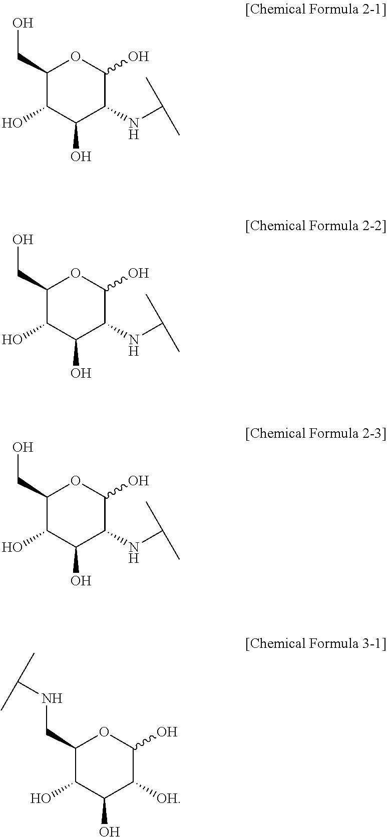

3. The compound or the hydrate thereof of claim 1, wherein in Chemical Formula 4 and Chemical Formula 5,
L¹ is independently of each other C₁₋₅ alkylene;
R¹ is independently of each other hydrogen, C₁₋₅ alkyl, or 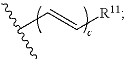
R² is independently of each other hydrogen, C₁₋₅ alkyl, or 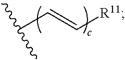
R¹¹ of R¹ and R² is independently of each other C₆₋₂₀ aryl, and the C₆₋₂₀ aryl may be substituted by one or more substituents selected from the group consisting of halogen, C₁₋₅ alkyl, and C₁₋₅ alkoxy;
c is independently of each other an integer of 1 to 5; and
a and b are independently of each other an integer of 1 to 3.

4. The compound or the hydrate thereof of claim 1, wherein R¹ and R² are independently of each other hydrogen, —CH₃,

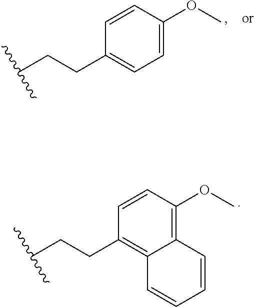 [SMILES: *CCc1ccc(OC)cc1 ; *CCc1ccc(OC)c2ccccc12]

5. The compound or the hydrate thereof of claim 1, wherein Chemical Formula 4 is a fluorophore represented by the following Chemical Formula 4-1, Chemical Formula 4-2, or Chemical Formula 4-3:

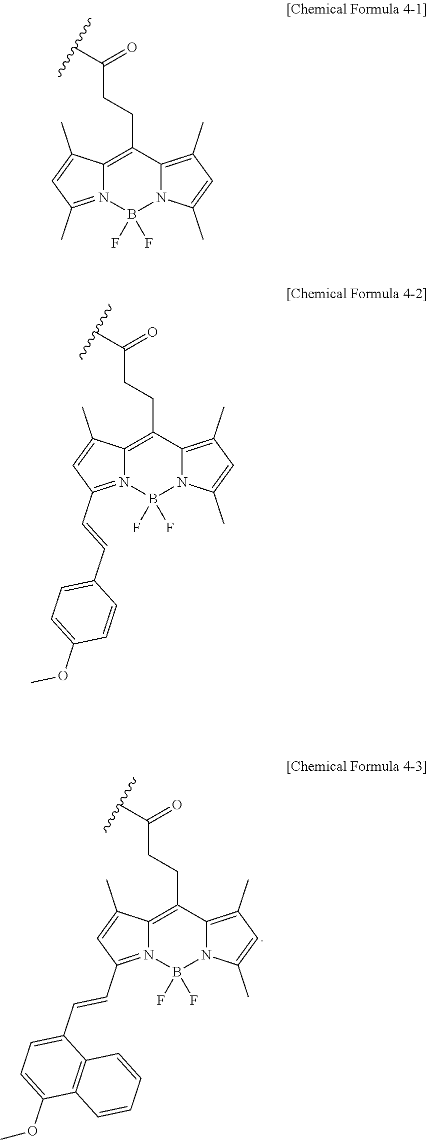

6. The compound or the hydrate thereof of claim 1, wherein Chemical Formula 5 is a fluorophore represented by the following Chemical Formula 5-1:

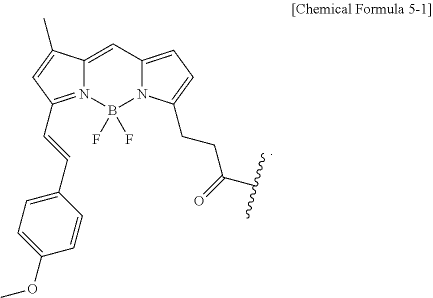

7. The compound or the hydrate thereof of claim 1, wherein in Chemical Formula 6 and Chemical Formula 7,
L² is independently of each other C₃₋₈ alkylene;
R³ and R⁴ are independently of each other hydrogen or C₁₋₅ alkyl;
R⁵ and R⁶ are independently of each other C₁₋₅ alkyl;
R⁷ is independently of each other C₁₋₅ alkyl; and
i is independently of each other an integer of 0 to 5.

8. The compound or the hydrate thereof of claim 1, wherein in Chemical Formula 6 and Chemical Formula 7,
R³ to R⁴ are hydrogen; and
R⁵ and R⁶ are —CH₃.

9. The compound or the hydrate thereof of claim 1, wherein Chemical Formula 6 is a fluorophore represented by the following Chemical Formula 6-1 or Chemical Formula 6-2:

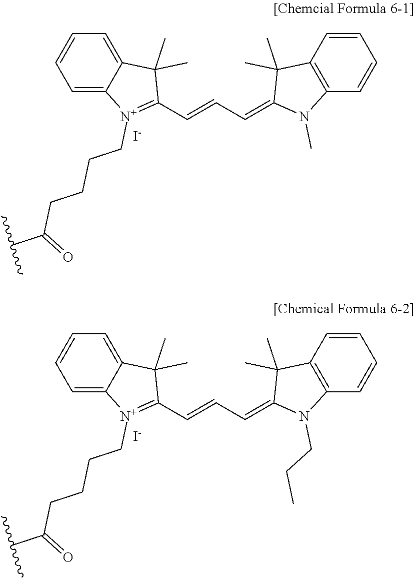 [SMILES: *C(=O)CCCC[N+]1=C(/C=C/C=C2/N(C)c3ccccc3C2(C)C)C(C)(C)c2ccccc21 ; [I-] ; *C(=O)CCCC[N+]1=C(/C=C/C=C2/N(CCC)c3ccccc3C2(C)C)C(C)(C)c2ccccc21 ; [I-]]

10. The compound or the hydrate thereof of claim 1, wherein Chemical Formula 7 is a fluorophore represented by the following Chemical Formula 7-1, Chemical Formula 7-2, or Chemical Formula 7-3:

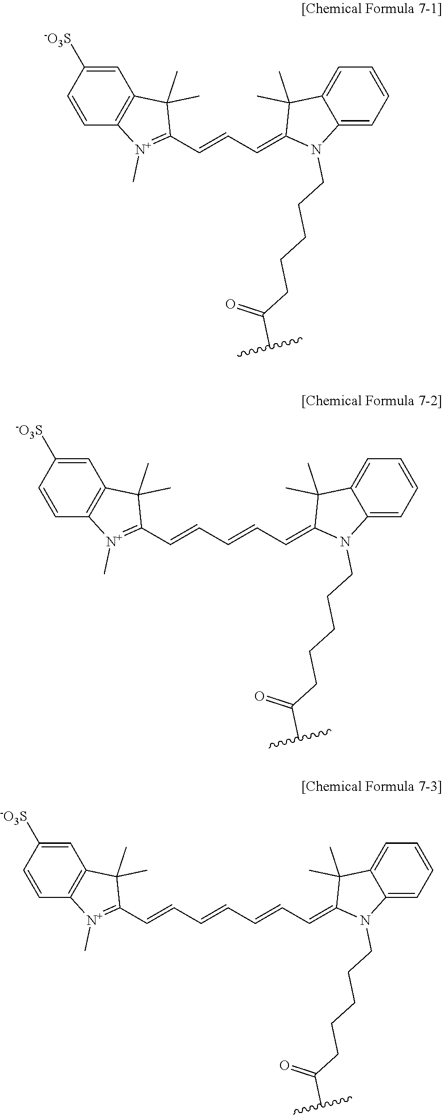

11. The compound or the hydrate thereof of claim 1, wherein the compound represented by Chemical Formula 1 is selected from the following compound group:

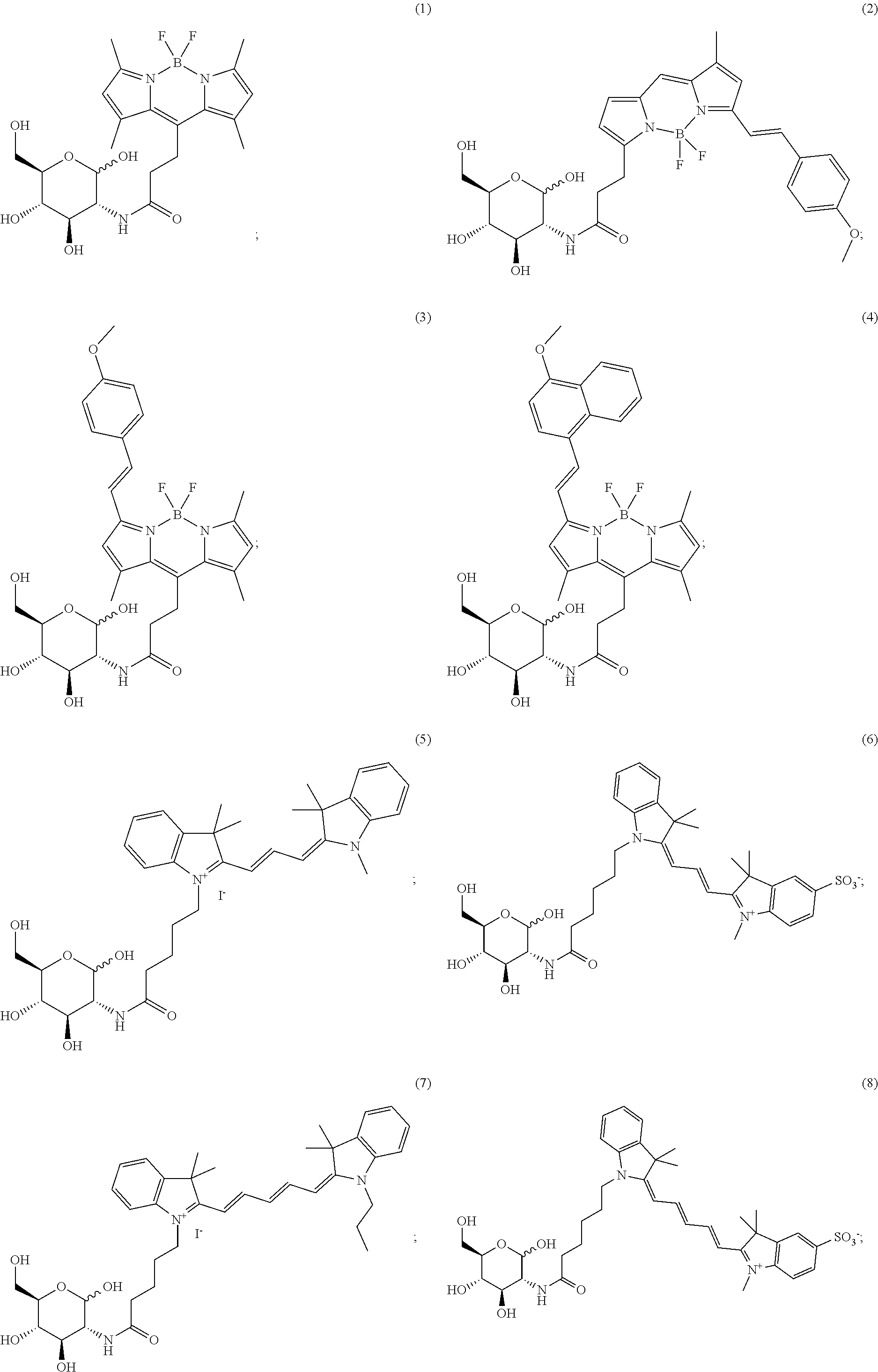

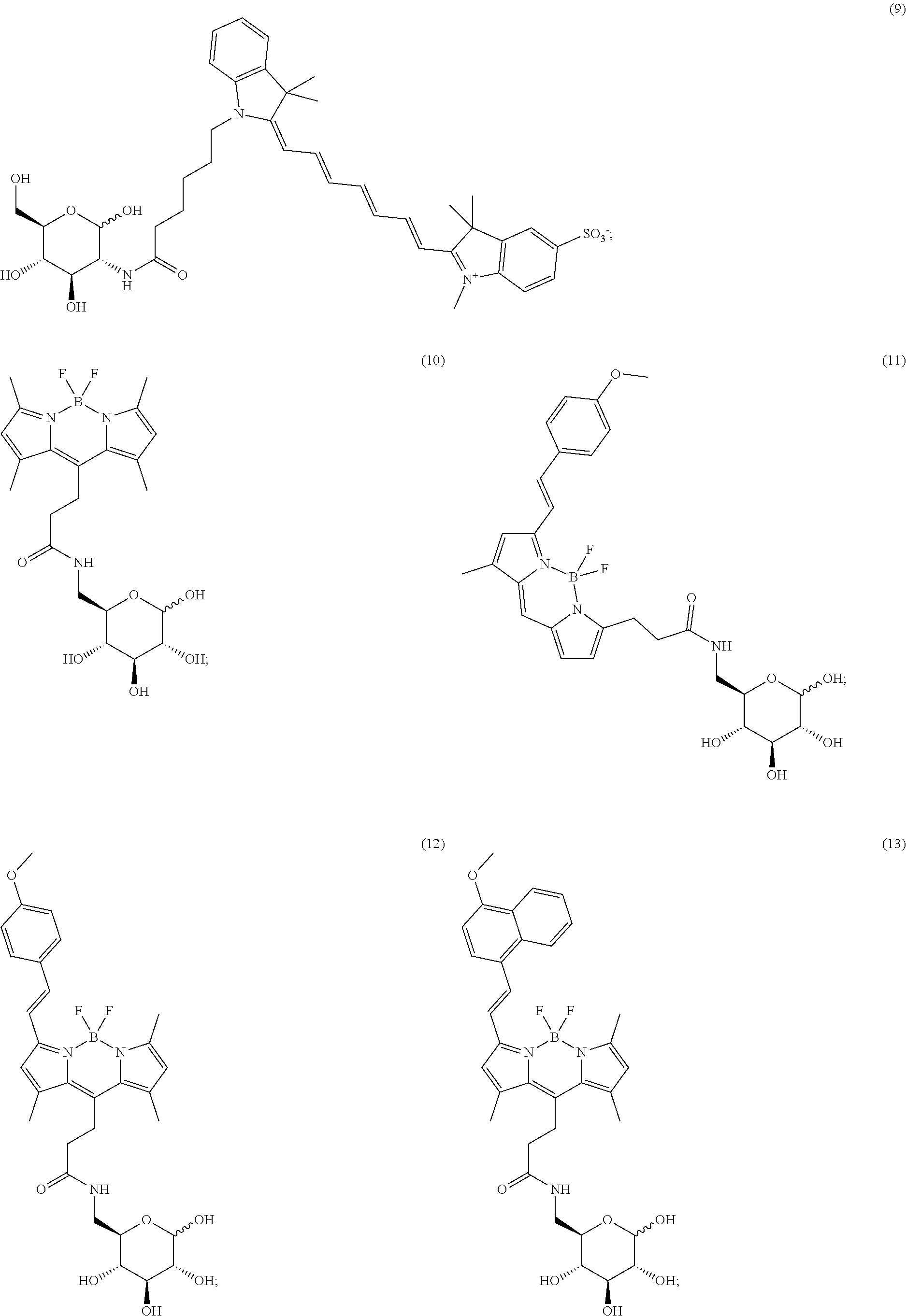

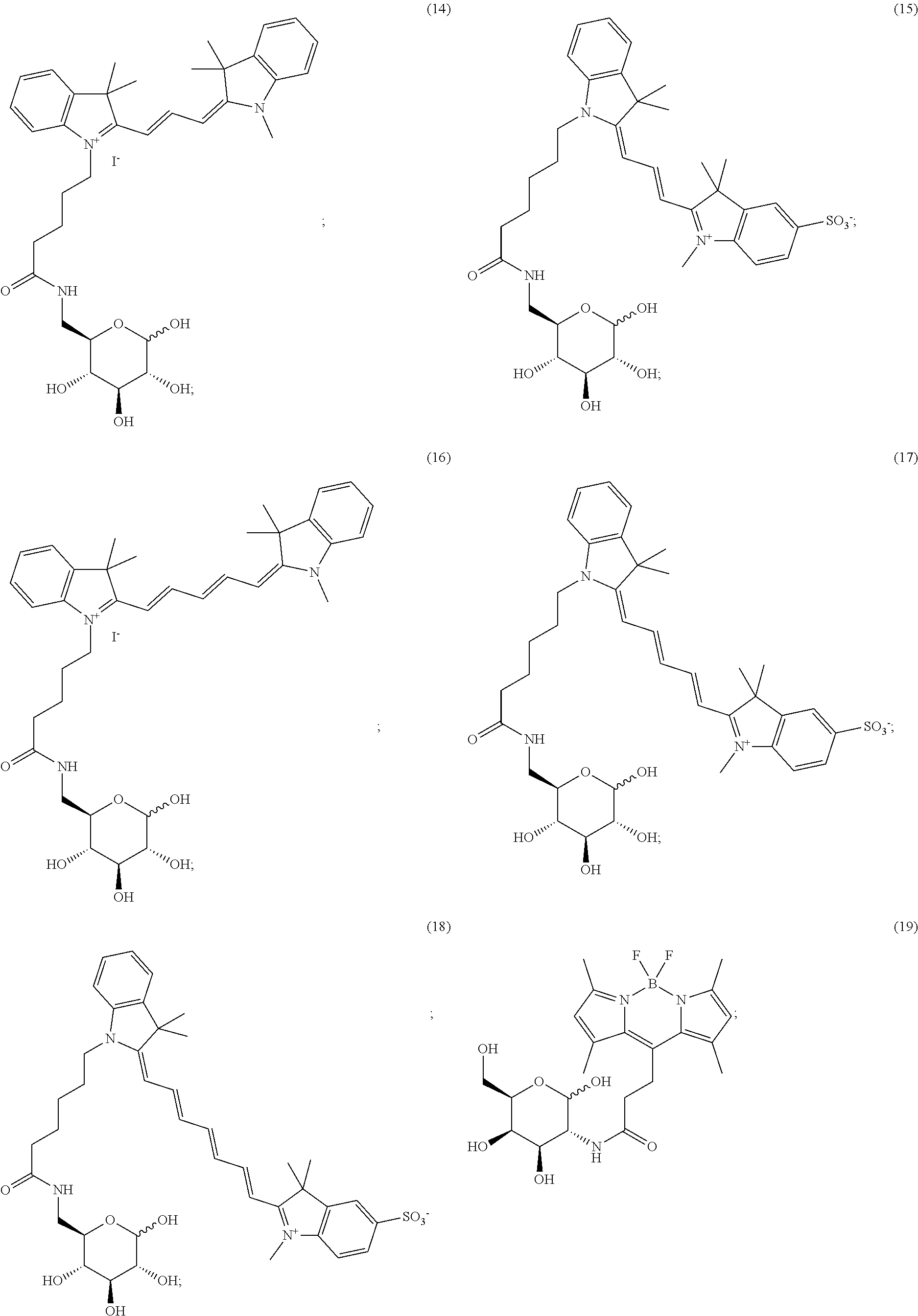

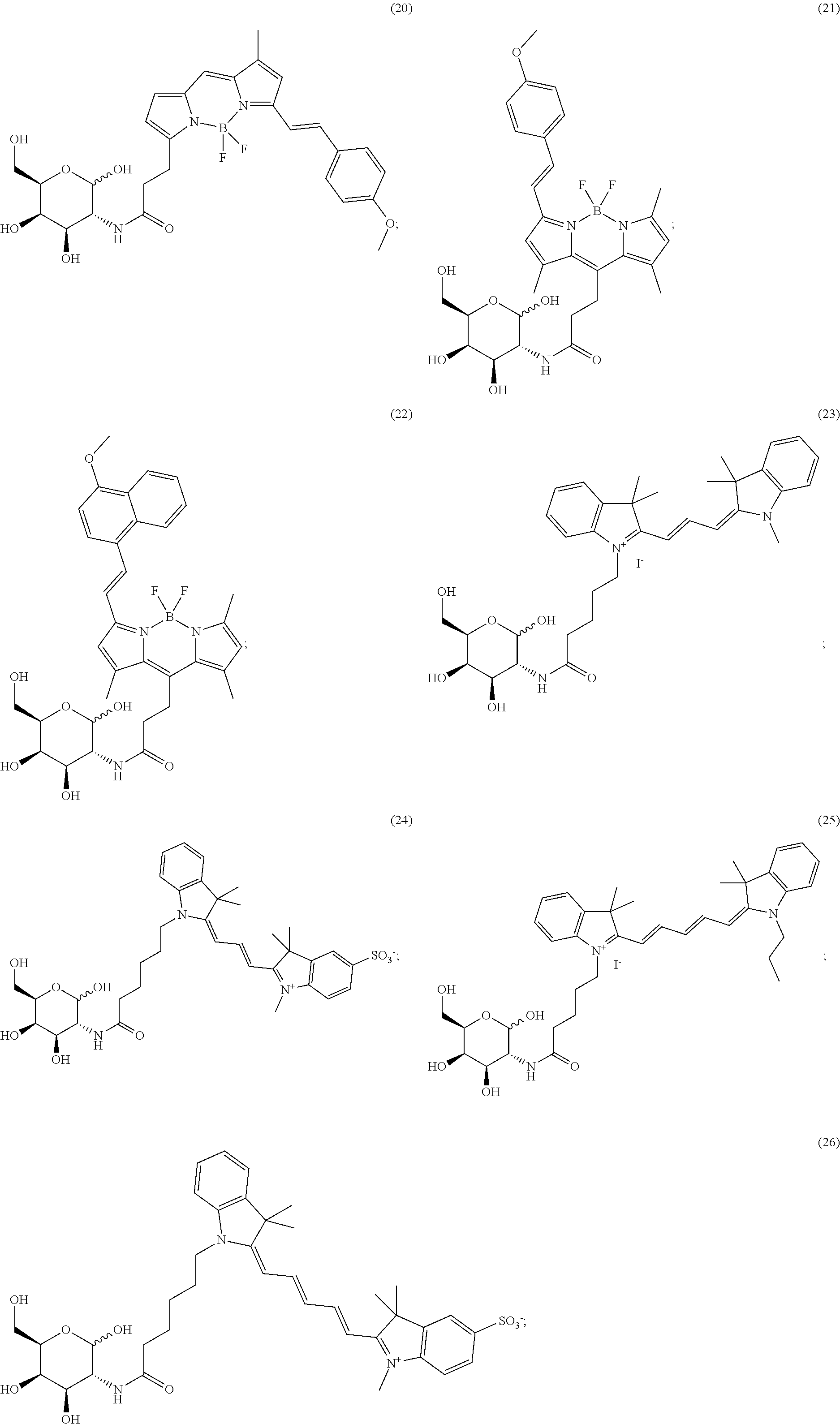

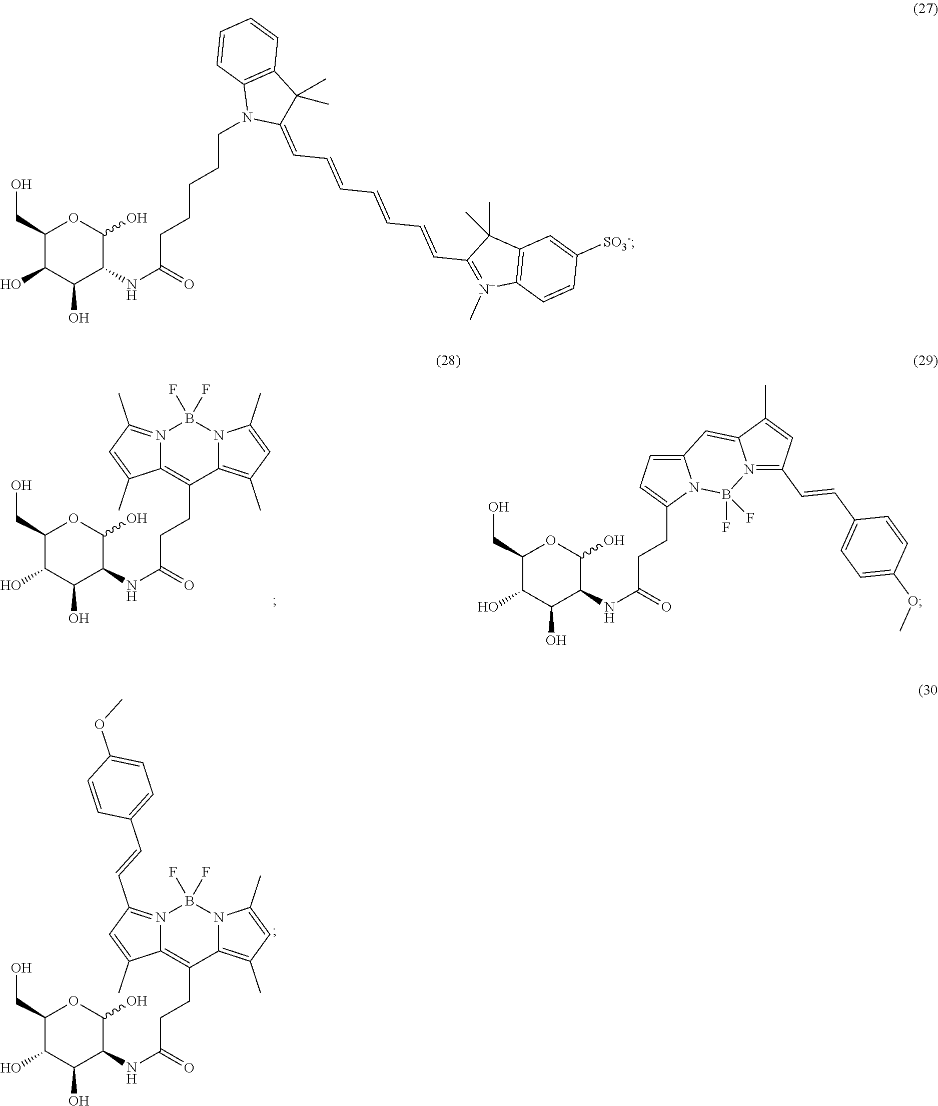

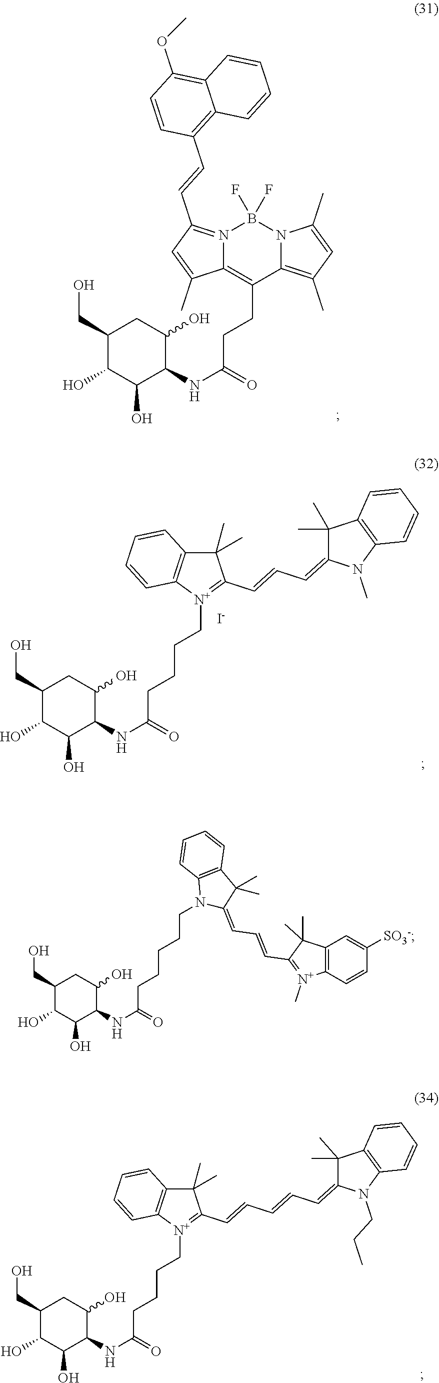

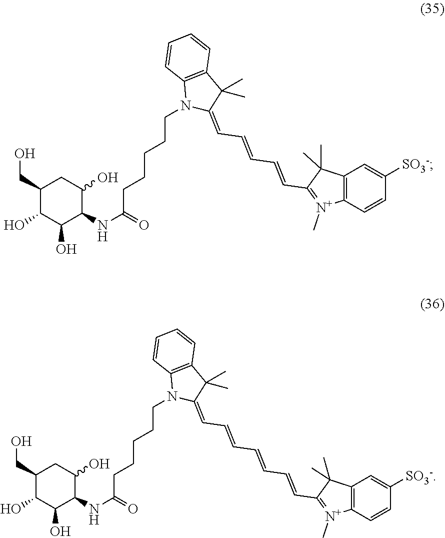

12. A fluorescent probe for labeling an M1 macrophage comprising a compound represented by the following Chemical Formula 1 or a hydrate thereof: 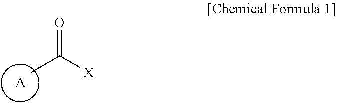
wherein
A is a carbohydrate derivative represented by the following Chemical Formula 2 or Chemical Formula 3: 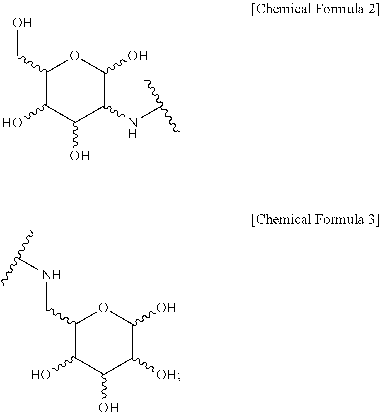 [SMILES: *NC1C(O)OC(CO)C(O)C1O ; *NCC1OC(O)C(O)C(O)C1O]
and
X is a fluorophore represented by any one of the following Chemical Formula 4 to Chemical Formula 7: 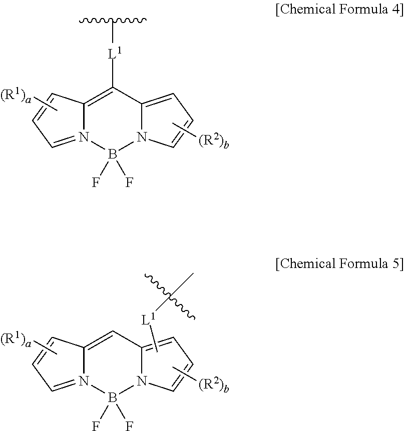
wherein
L¹ is independently of each other C₁₋₁₀ alkylene;
R¹ is independently of each other hydrogen, C₁₋₁₀ alkyl or 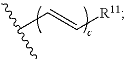
R² is independently of each other hydrogen, C₁₋₁₀ alkyl, or 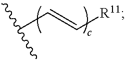
R¹¹ of R¹ and R² is independently of each other C₆₋₂₀ aryl, and the C₆₋₂₀ aryl may be substituted by one or more substituents selected from the group consisting of halogen, C₁₋₅ alkyl, and C₁₋₅ alkoxy;
c is independently of each other an integer of 0 to 10; and
a and b are independently of each other an integer of 1 to 3; 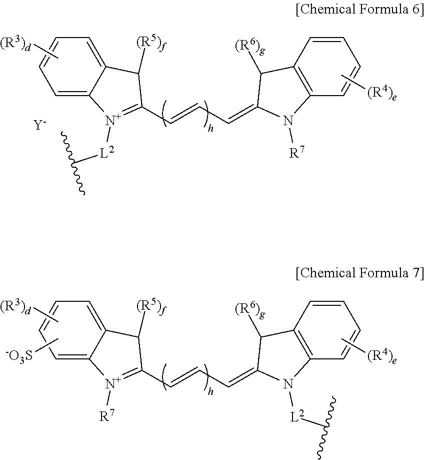
wherein
L² is independently of each other C₁₋₁₀ alkylene;
R³ and R⁴ are independently of each other hydrogen, halogen, C₁₋₁₀ alkyl, or C₁₋₁₀ alkoxy;
R⁵ and R⁶ are independently of each other hydrogen or C₁₋₁₀ alkyl;
R⁷ is independently of each other hydrogen or C₁₋₁₀ alkyl;
d is independently of each other an integer of 1 to 3;
e is independently of each other an integer of 1 to 4;
f and g are independently of each other 1 or 2;
h is independently of each other an integer of 0 to 10; and
Y⁻ is an anion.

13. The fluorescent probe for labeling an M1 macrophage of claim 12, wherein the fluorescent probe for labeling an M1 macrophage targets glucose transporters (GLUTs).

14. The fluorescent probe for labeling an M1 macrophage of claim 13, wherein the glucose transporter is GLUT1.

15. A composition for visualization of an M1 macrophage comprising the fluorescent probe for labeling an M1 macrophage according to claim 12.

16. A kit for visualization of an M1 macrophage comprising the fluorescent probe for labeling an M1 macrophage according to claim 12.

17. An M1 macrophage monitoring system comprising the fluorescent probe for labeling an M1 macrophage of any one of claim 12.

18. A method for providing information on an M1 macrophage by administering a composition comprising the fluorescent probe for labeling an M1 macrophage according to claim 12 to a biological sample.

19. A method for providing information needed to diagnose an inflammatory disease, the method comprising obtaining a fluorescence image by bringing a biological sample separated from a subject into contact with the fluorescent probe for labeling an M1 macrophage according to claim 12.

20. The method for providing information needed to diagnose an inflammatory disease of claim 19, wherein the inflammatory disease comprises an autoimmune disease.
